(19) United States
(12) Patent Application Publication
(10) Pub. No.: US 2026/0048204 A1
(43) Pub. Date: Feb. 19, 2026

(54) MODULAR DELIVERY PEN SYSTEMS AND METHODS OF USING THE SAME

(71) Applicant: Embecta Corp., Parsippany, NJ (US)

(72) Inventors: Andrew Barnett, Andover, MA (US); Eugene Prais, Andover, MA (US)

(73) Assignee: Embecta Corp., Parsippany, NJ (US)

(21) Appl. No.: 19/101,184

(22) PCT Filed: Aug. 16, 2023
(86) PCT No.: PCT/US2023/072272

§ 371 Date: Feb. 4, 2025

Related U.S. Application Data
(60) Provisional application No. 63/371,540, filed on Aug. 16, 2022.

(51) Int. Cl. A61M 5/315; A61M 5/20; A61M 5/31

(52) U.S. Cl. CPC A61M 5/31551; A61M 5/2033; A61M 5/31568; A61M 5/3158; A61M 2005/3126; A61M 2205/3306; A61M 2205/3317; A61M 2205/3327

(57) ABSTRACT

A dosing module includes an outer shell having a stem and a spline hole plate having a spline hole, where the spline hole plate is positioned within the stem and sized to receive a spline shaft extending from a shaft spring, such that the spline hole plate and the shaft spring rotate together. The dosing module includes an encoder wheel coupled to the spline hole plate and positioned within the stem and sensors positioned within the stem configured to detect rotation of the encoder wheel. The dosing module is removably couplable to a dose knob of a delivery pen, where the stem is received within an internal well of the dose knob. The shaft spring is directly coupled to a component of a drive mechanism of the delivery pen, and the dosing module is configured to measure a size of a dose administered from the delivery pen.

### CROSS-REFERENCE TO RELATED APPLICATION

This application claims the benefit of and priority to U.S. Provisional Patent Application No. 63/371,540, filed Aug. 16, 2022, the contents of which are incorporated herein by reference in their entirety.

### FIELD

This disclosure relates to modular delivery pen systems and methods of using modular delivery pen systems.

### BACKGROUND

For treatment of some diseases and conditions, it is often desirable to inject medication directly into the tissue of a patient. For instance, syringes or pen injection devices are used to inject medicaments into tissue areas, such as the intramuscular tissue layer, the subcutaneous tissue layer, and the intradermal tissue layer. It is often difficult for health care workers or patients self-administering the medication via the syringe or pen injection device to accurately determine the amount of medicament administered into the tissue via the syringe or pen injection. Therefore, there is a need for medicament delivery systems that allow for precise and accurate determination of an amount of medicament administered to a patient.

Many syringes or pen injection devices are single use. The syringes and pen injection devices in the industry are typically disposed of after one, or just several, injections. Therefore, there is also a need for medicament delivery systems that allow for precise and accurate determination of an amount of medicament administered to a patient, where the capital equipment used to determine the dose amount administered to a patient is modular from the syringe or pen injection device, thereby allowing the syringe or pen injection device to be disposed of as needed and allowing the capital equipment to be used throughout its full lifetime with multiple different syringes or pen injection devices.

Improved delivery pen systems and methods of using the same are desired.

### SUMMARY

The present disclosure relates to a dosing module, including: an outer shell, the outer shell including a stem; a spline hole plate having a spline hole, wherein the spline hole plate is positioned within the stem of the outer shell and sized to receive a spline shaft extending proximally from a shaft spring, such that the spline hole plate and the shaft spring are rotatable together; an encoder wheel rigidly coupled to the spline hole plate and positioned within the stem of the outer shell; one or more sensors positioned within the stem of the outer shell and configured to detect a rotation of the encoder wheel, wherein the encoder wheel is configured to rotate with respect to the one or more sensors, wherein: the dosing module is removably couplable to a dose knob of a delivery pen, wherein the stem is received within an internal well of the dose knob when the dosing module is coupled to the dose knob; the shaft spring is directly coupled to a component of a drive mechanism of the delivery pen; and the dosing module is configured to measure a size of a dose administered from the delivery pen.

In some embodiments, the present disclosure relates to a dosing module, wherein the dosing module is removably couplable to the dose knob when a first button originally coupled to the delivery pen is removed from the delivery pen. In some embodiments, the present disclosure relates to a dosing module, where the first button, when coupled to the delivery pen, is configured to initiate administration of a dose from the delivery pen when the first button is depressed. In some embodiments, the present disclosure relates to a dosing module, wherein: the stem has a peripheral edge; and the peripheral edge of the stem is configured to contact a radially inner surface of the internal well when the dosing module is coupled to the dose knob. In some embodiments, the present disclosure relates to a dosing module, wherein: the stem includes a peripheral surface and a retaining ridge extending radially outward from the peripheral surface of the stem; and the retaining ridge is configured to be received in a groove along a radially inner surface of the internal well when the dosing module is coupled to the dose knob. In some embodiments, the present disclosure relates to a dosing module, wherein: the outer shell further includes a body having a peripheral edge; and an outer dimension of the peripheral edge of the body of the outer shell is equal to or less than an outer dimension of a peripheral edge of the dose knob. In some embodiments, the present disclosure relates to a dosing module, wherein the outer dimension of the peripheral edge of the body of the outer shell is greater than an outer dimension of a peripheral edge of the stem of the outer shell. In some embodiments, the present disclosure relates to a dosing module, further including: a button; a circuit board; and one or more conductive spring clips, wherein each of the one or more conductive spring clips have a first end coupled to the circuit board and a second free end, wherein: the button is configured to initiate administration of a dose from the delivery pen when the button is depressed; and when the button is depressed, the button is configured to deform the one or more conductive spring clips such that the one or more conductive spring clips complete a circuit. In some embodiments, the present disclosure relates to a dosing module, further including: an inner shell positioned within an interior of the outer shell, wherein: the spline hole plate is coupled to the outer shell such that the spline hole plate and the outer shell are rotatable together; the button is positioned at least partially within the inner shell; and the outer shell is rotatable relative to the inner shell.

The present disclosure relates to a dosing module, including: an outer shell, the outer shell including a body and a stem, wherein: the stem is sized to be received within an internal well of a dose knob of a delivery pen; and an outer dimension of a peripheral edge of the body of the outer shell is equal to or less than an outer dimension of a peripheral edge of the dose knob, wherein: the dosing module is removably couplable to the dose knob of the delivery pen; and the dosing module is configured to measure a size of a dose administered from the delivery pen.

In some embodiments, the present disclosure relates to a dosing module, wherein the outer dimension of the peripheral edge of the body of the outer shell is greater than an outer dimension of a peripheral edge of the stem of the outer shell. In some embodiments, the present disclosure relates to a dosing module, wherein the body of the outer shell further includes a distal end wall having a distal surface, wherein the distal surface of the distal end wall is configured to contact a proximal surface of a proximal end wall of the dose knob when the dosing module is coupled to the dose knob. In some embodiments, the present disclosure relates to a dosing module, wherein: the stem includes a peripheral edge; and the peripheral edge of the stem is configured to contact a radially inner surface of the internal well when the dosing module is coupled to the dose knob. In some embodiments, the present disclosure relates to a dosing module, wherein: the stem includes a peripheral surface and a retaining ridge extending radially outward from the peripheral surface of the stem; and the retaining ridge is configured to be received in a groove along a radially inner surface of the internal well when the dosing module is coupled to the dose knob. In some embodiments, the present disclosure relates to a dosing module, wherein the stem includes a distal wall having an opening configured to receive a shaft coupled to a drive mechanism of the delivery pen. In some embodiments, the present disclosure relates to a dosing module, wherein the opening in the distal wall of the stem is chamfered or beveled. In some embodiments, the present disclosure relates to a dosing module, further including a sensor coupled to the stem, wherein the sensor is configured to detect when the stem is received within the internal well.

The present disclosure relates to a dosing module, including: a spline hole plate having a spline hole, wherein the spline hole is sized to receive a spline shaft extending proximally from a shaft spring, such that the spline hole plate and the shaft spring are rotatable together; and an encoder wheel rigidly coupled to the spline hole plate, wherein: the shaft spring is coupled to a component of a drive mechanism of a delivery pen; the dosing module is removably couplable to the delivery pen; and the dosing module is configured to measure a size of a dose administered from the delivery pen.

In some embodiments, the present disclosure relates to a dosing module, further including one or more sensors configured to detect a rotation of the encoder wheel, wherein the encoder wheel is configured to rotate with respect to the one or more sensors. In some embodiments, the present disclosure relates to a dosing module, wherein: the encoder wheel includes one or more electrically conductive pads; and the one or more sensors include contact clips configured to contact the one or more electrically conductive pads. In some embodiments, the present disclosure relates to a dosing module, wherein: the encoder wheel includes one or more magnetic poles; and the one or more sensors include one or more magnetic sensors. In some embodiments, the present disclosure relates to a dosing module, wherein: the encoder wheel includes one or more markings; and the one or more sensors include one or more optical sensors. In some embodiments, the present disclosure relates to a dosing module, further including a button, wherein the button is configured to initiate administration of a dose from the delivery pen when the button is depressed. In some embodiments, the present disclosure relates to a dosing module, further including: a circuit board; and one or more conductive spring clips, wherein each of the one or more conductive spring clips have a first end coupled to the circuit board and a second free end, wherein: when the button is depressed, the button is configured to deform the one or more conductive spring clips such that the one or more conductive spring clips complete a circuit. In some embodiments, the present disclosure relates to a dosing module, further including: an outer shell; and an inner shell positioned within an interior of the outer shell, wherein: the spline hole plate is coupled to the outer shell such that the spline hole plate and the outer shell are rotatable together; the button is positioned at least partially within the inner shell; and the outer shell is rotatable relative to the inner shell.

The present disclosure relates to a delivery pen system, including: a delivery pen, including: a dose knob at a proximal end of the delivery pen, wherein the dose knob includes an internal well; and a proximally extending spline shaft positioned in the internal well; and a dosing module removably couplable to the dose knob and configured to measure a size of a dose administered from the delivery pen, wherein the dosing module includes a spline hole plate having a spline hole sized to receive the proximally extending spline shaft such that the spline hole plate and the spline shaft are rotatable together.

In some embodiments, the present disclosure relates to a delivery pen system, wherein the delivery pen further includes: a drive mechanism for delivering a dose, the drive mechanism including an inner sleeve; and a shaft spring positioned in the internal well, wherein: the spline shaft extends proximally from the shaft spring; and the inner sleeve is coupled to the shaft spring such that the inner sleeve and the shaft spring are rotatable together. In some embodiments, the present disclosure relates to a delivery pen system, wherein at least a portion of the inner sleeve is positioned in the internal well. In some embodiments, the present disclosure relates to a delivery pen system, wherein: a proximal surface of a distal end wall of the dose knob and a distal surface of a flange of the inner sleeve include corresponding features that are configured to mate to allow the dose knob and the inner sleeve to rotate together. In some embodiments, the present disclosure relates to a delivery pen system, wherein the corresponding features include corresponding ribbing. In some embodiments, the present disclosure relates to a delivery pen system, wherein the inner sleeve and the shaft spring include corresponding features that are configured to mate to allow the inner sleeve and the shaft spring to rotate together. In some embodiments, the present disclosure relates to a delivery pen system, wherein the corresponding features include: one or more pegs extending proximally from a flange of the inner sleeve; and one or more bores in a surface of the shaft spring, the one or more bores configured to receive the one or more pegs. In some embodiments, the present disclosure relates to a delivery pen system, wherein the spline hole is chamfered or beveled. In some embodiments, the present disclosure relates to a delivery pen system, wherein the dosing module further includes: an encoder wheel coupled to the spline hole plate such that the encoder wheel and spline hole plate are rotatable together; and one or more sensors configured to detect a rotation of the encoder wheel. In some embodiments, the present disclosure relates to a delivery pen system, wherein: the encoder wheel includes one or more electrically conductive pads; and the one or more sensors include contact clips configured to contact the one or more electrically conductive pads, wherein the encoder wheel is configured to rotate relative the contact clips. In some embodiments, the present disclosure relates to a delivery pen system, wherein: the encoder wheel includes one or more magnetic poles; and the one or more sensors include one or more magnetic sensors, wherein the encoder wheel is configured to rotate relative the one or more magnetic sensors. In some embodiments, the present disclosure relates to a delivery pen system, wherein: the encoder wheel includes one or more markings; and the one or more sensors include one or more optical sensors, wherein the encoder wheel is configured to rotate with respect to the one or more optical sensors. In some embodiments, the present disclosure relates to a delivery pen system, wherein the dosing module further includes a button, wherein the button is configured to initiate administration of a dose from the delivery pen when the button is depressed. In some embodiments, the present disclosure relates to a delivery pen system, wherein the dosing module further includes: a circuit board; and one or more conductive spring clips, wherein each of the one or more conductive spring clips have a first end coupled to the circuit board and a second free end, wherein: when the button is depressed, the button deforms the one or more conductive spring clips such the one or more conductive spring clips complete a circuit. In some embodiments, the present disclosure relates to a delivery pen system, further including: an outer shell; and an inner shell positioned within an interior of the outer shell, wherein: the spline hole plate is coupled to the outer shell such that the spline hole plate and the outer shell are rotatable together; the button is positioned at least partially within the inner shell; and the outer shell is rotatable relative to the inner shell. In some embodiments, the present disclosure relates to a delivery pen system, further including a button removably couplable to the dose knob, wherein the button, when coupled to the delivery pen, is configured to initiate administration of a dose from the delivery pen when the button is depressed. In some embodiments, the present disclosure relates to a delivery pen system, wherein the dosing module is couplable to the dose knob when the button is removed from the dose knob.

The present disclosure relates to a delivery pen system, including: a delivery pen, including a dose knob at a proximal end of the delivery pen, wherein the dose knob includes: an internal well; and a peripheral edge; and a dosing module removably couplable to the dose knob and configured to measure a size of a dose administered from the delivery pen, wherein the dosing module includes an outer shell, the outer shell including a body and a stem, wherein: the stem is sized to be received within the internal well; and an outer dimension of a peripheral edge of the body of the outer shell is equal to or less than an outer dimension of the peripheral edge of the dose knob.

In some embodiments, the present disclosure relates to a delivery pen system, wherein the outer dimension of the peripheral edge of the body of the outer shell is greater than an outer dimension of a peripheral edge of the stem of the outer shell. In some embodiments, the present disclosure relates to a delivery pen system, wherein: the dose knob includes a proximal end wall having a proximal surface; the body of the outer shell of the dosing module includes a distal end wall having a distal surface; and the distal surface of the distal end wall of the body is configured to contact the proximal surface of the proximal end wall of the dose knob when the dosing module is coupled to the dose knob. In some embodiments, the present disclosure relates to a delivery pen system, wherein: the internal well includes a radially inner surface; the stem includes a peripheral edge; and the radially inner surface of the internal well is configured to contact the peripheral edge of the stem when the dosing module is coupled to the dose knob. In some embodiments, the present disclosure relates to a delivery pen system, wherein contact between the radially inner surface of the internal well and the peripheral edge of the stem when the dosing module is coupled to the dose knob is configured to create a friction fit between the dosing module and the dose knob. In some embodiments, the present disclosure relates to a delivery pen system, wherein: the internal well includes a radially inner surface and a groove along the radially inner surface; the stem includes a peripheral surface and a retaining ridge extending radially outward from the peripheral surface of the stem; and the retaining ridge is configured to be received in the groove when the dosing module is coupled to the dose knob. In some embodiments, the present disclosure relates to a delivery pen system, wherein the retaining ridge and the groove are configured to form a snap fit when the dosing module is coupled to the dose knob. In some embodiments, the present disclosure relates to a delivery pen system, wherein: the dose knob includes a shaft coupled to a drive mechanism of the delivery pen; the stem includes a distal wall having an opening; and the shaft is received in the opening in the distal wall of the stem when the dosing module is coupled to the dose knob. In some embodiments, the present disclosure relates to a delivery pen system, wherein the opening in the distal wall of the stem is chamfered or beveled. In some embodiments, the present disclosure relates to a delivery pen system, wherein the dosing module further includes a sensor coupled to the stem, wherein the sensor is configured to detect when the stem is received within the internal well. In some embodiments, the present disclosure relates to a delivery pen system, further including a button removably couplable to the dose knob, wherein the button, when coupled to the delivery pen, is configured to initiate administration of a dose from the delivery pen when the button is depressed. In some embodiments, the present disclosure relates to a delivery pen system, wherein the dosing module is couplable to the dose knob when the button is removed from the dose knob.

The present disclosure relates to a delivery pen system, including: a delivery pen, including: a dose knob at a proximal end of the delivery pen, wherein the dose knob includes an internal well and a proximal surface at a distal end wall of the internal well; a shaft spring positioned in the internal well, the shaft spring including a spline shaft extending proximally therefrom; and a drive mechanism for delivering a dose, the drive mechanism including an inner sleeve including a flange having a proximal surface and a distal surface, wherein: the proximal surface of the flange of the inner sleeve and the shaft spring include corresponding features that are configured to mate to allow the inner sleeve and the shaft spring to rotate together; and the proximal surface of the distal end wall of the internal well and the distal surface of the flange of the inner sleeve include corresponding features that are configured to mate to allow the dose knob and the inner sleeve to rotate together; and a dosing module removably couplable to the dose knob and configured to measure a size of a dose administered from the delivery pen, wherein the dosing module includes: a spline hole plate having a spline hole sized to receive the spline shaft such that the spline hole plate and the spline shaft are rotatable together; an encoder wheel coupled to the spline hole plate such that the encoder wheel and spline hole plate are rotatable together, wherein the encoder wheel includes one or more electrically conductive pads; and one or more contact clips configured to contact the one or more electrically conductive pads, wherein the encoder wheel is configured to rotate relative the one or more contact clips.

The present disclosure relates to a delivery pen system, including: a delivery pen, including a dose knob at a proximal end of the delivery pen; a button removably couplable to the dose knob; and a dosing module removably couplable to the dose knob and configured to measure a size of a dose administered from the delivery pen.

The present disclosure relates to a system, including: a delivery pen, including: a dose knob at a proximal end of the delivery pen, wherein the dose knob includes an internal well; a drive mechanism for delivering a dose; and a proximally extending spline shaft positioned in the internal well, wherein the spline shaft is coupled to a component of the drive mechanism such that the spline shaft and the component of the drive mechanism are rotatable together; a dosing module removably couplable to the dose knob, wherein the dosing module includes: a spline hole plate having a spline hole sized to receive the proximally extending spline shaft such that the spline hole plate and the spline shaft are rotatable together; an encoder wheel coupled to the spline hole plate such that the encoder wheel and spline hole plate are rotatable together; and one or more sensors configured to sense a rotation of the encoder wheel, wherein the encoder wheel is configured to rotate with respect to the one or more sensors; and a control system configured to: receive sensor data from the one or more sensors; determine a degree of rotation of the encoder wheel based on the sensor data; and determine a size of a dose delivered from the delivery pen during an injection.

In some embodiments, the present disclosure relates to a system, wherein: the encoder wheel includes one or more electrically conductive pads; and the one or more sensors include contact clips configured to contact the one or more electrically conductive pads. In some embodiments, the present disclosure relates to a system, wherein: the encoder wheel includes one or more magnetic poles; and the one or more sensors include one or more magnetic sensors. In some embodiments, the present disclosure relates to a system, wherein: the encoder wheel includes one or more markings; and the one or more sensors include one or more optical sensors. In some embodiments, the present disclosure relates to a system, wherein the control system is further configured to determine the size of the dose delivered from the delivery pen during the injection based, at least in part, on the degree of rotation of the encoder wheel. In some embodiments, the present disclosure relates to a system, wherein the control system is further configured to determine a degree of rotation of one or more components of the drive mechanism of the delivery pen based on the degree of rotation of the encoder wheel. In some embodiments, the present disclosure relates to a system, wherein the control system is further configured to determine the size of the dose delivered from the delivery pen during the injection based, at least in part, on the degree of rotation of the one or more components of the drive mechanism. In some embodiments, the present disclosure relates to a system, wherein: the dosing module further includes: a button configured to initiate administration of a dose from the delivery pen when the button is depressed; a circuit board; and one or more conductive spring clips, wherein each of the one or more conductive spring clips have a first end coupled to the circuit board and a second free end, wherein: when the button is depressed, the button is configured to deform the one or more conductive spring clips such that the one or more conductive spring clips complete a circuit; and the control system is further configured to: receive a signal indicating the circuit is complete; and determine that the injection has been initiated based on the signal indicating the circuit is complete. In some embodiments, the present disclosure relates to a system, wherein the control system is further configured to: receive sensor data from the one or more sensors and determine the degree of rotation of the encoder wheel based on the sensor data for as long as the circuit is complete. In some embodiments, the present disclosure relates to a system, wherein the control system is further configured to: receive sensor data from the one or more sensors and determine the degree of rotation of the encoder wheel based on the sensor data for a set period of time after receiving the signal indicating the circuit is complete. In some embodiments, the present disclosure relates to a system, wherein the control system is further configured to display the size of the dose delivered on a display of the dosing module. In some embodiments, the present disclosure relates to a system, wherein the control system is further configured to display the size of the dose delivered on a display external to the dosing module. In some embodiments, the present disclosure relates to a system, wherein the control system is internal to the dosing module. In some embodiments, the present disclosure relates to a system, wherein: the dosing module further includes a second sensor configured to sense when the dosing module is received in the internal well of the dose knob; and the control system is further configured to: receive sensor data from the second sensor; and determine when the dosing module was last coupled to a new delivery pen based on the sensor data from the second sensor. In some embodiments, the present disclosure relates to a system, wherein: the dosing module further includes a second sensor configured to sense when the dosing module is received in the internal well of the dose knob; and the control system is further configured to: receive sensor data from the second sensor; and determine an amount of medicament left in the delivery pen based on the sensor data from the second sensor and the size of the dose delivered from the delivery pen during the injection.

The present disclosure relates to a method, including: sensing, with at least one sensor of a dosing module, data regarding a rotation of an encoder wheel of the dosing module, wherein: the encoder wheel is rotatable with a spline shaft of a delivery pen; and the spline shaft is coupled to a component of a drive mechanism of the delivery pen, wherein the drive mechanism is configured to deliver a dose from the delivery pen; sending, by the at least one sensor, the sensed data to a controller; identifying, by the controller and from the sensed data, a degree of rotation of the encoder wheel; and identifying, by the controller, a size of a dose delivered from the delivery pen during an injection.

In some embodiments, the present disclosure relates to a method, wherein the size of the dose delivered from the delivery pen during the injection is determined from the degree of rotation of the encoder wheel. In some embodiments, the present disclosure relates to a method, further including: identifying, by the controller and from the degree of rotation of the encoder wheel, a degree of rotation of one or more components of the drive mechanism. In some embodiments, the present disclosure relates to a method, wherein the size of the dose delivered from the delivery pen during the injection is determined from the degree of rotation of the one or more components of the drive mechanism. In some embodiments, the present disclosure relates to a method, further including displaying the size of the dose delivered from the delivery pen during the injection on a display of the dosing module. In some embodiments, the present disclosure relates to a method, further including displaying the size of the dose delivered from the delivery pen during the injection on a display external to the dosing module. In some embodiments, the present disclosure relates to a method, further including: identifying, by the controller, a time when the injection of the dose is initiated; and identifying, by the controller, the degree of rotation of the encoder wheel for a set period of time after the injection of the dose is initiated. In some embodiments, the present disclosure relates to a method, further including: identifying, by the controller, when a button of the dosing module is depressed, wherein the button is configured to initiate administration of the dose from the delivery pen when the button is depressed; and identifying, by the controller, the degree of rotation of the encoder wheel for as long as the button is depressed. In some embodiments, the present disclosure relates to a method, further including: sensing, with a second sensor of the dosing module, data regarding a coupling of the dosing module to the delivery pen; sending, by the second sensor, the sensed data to the controller; and identifying, by the controller and from the sensed data from the second sensor and the size of the dose delivered from the delivery pen during the injection, an amount of medicament left in the delivery pen.

### BRIEF DESCRIPTION OF DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 shows a schematic of an exemplary embodiment of a delivery pen system;

FIG. 2 shows an exploded view schematic of an exemplary embodiment of a delivery pen of the delivery pen system;

FIG. 3A depicts a perspective view of an exemplary embodiment of dose knob of a delivery pen with a button positioned at a proximal end of the dose knob;

FIG. 3B depicts an exploded view of the dose knob and the button;

FIG. 3C depicts an exploded view of the dose knob with the button removed;

FIG. 3D depicts a cross-sectional view of the dose knob with the button removed;

FIG. 3E depicts a perspective view of the dose knob with the well of the dose knob empty;

FIG. 3F depicts a perspective view of a proximal end of an inner sleeve;

FIG. 3G depicts a perspective view of the dose knob;

FIG. 4 depicts a side view of a dosing module;

FIG. 5 depicts a cross-sectional view of the dosing module;

FIG. 6 depicts a perspective view of the dosing module coupled to the delivery pen;

FIG. 7 depicts a cross-sectional view of the dosing module coupled to the delivery pen;

FIG. 8 depicts a cross-sectional view of the delivery pen system with the dosing module coupled to the delivery pen;

FIG. 9 depicts an operations system for operating the dosing module; and

FIGS. 10A-10C show a schematic of an exemplary embodiment of a delivery pen system.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments. Embodiment examples are described as follows with reference to the figures. Identical, similar, or identically acting elements in the various figures are identified with identical reference numbers and a repeated description of these elements is omitted in part to avoid redundancies. “Distal,” as used herein, refers to the direction toward or nearer a patient end of a delivery pen (i.e., a needle for injection) or other device. “Proximal,” as used herein, refers to the direction toward or nearer a user end of the delivery pen (i.e., a user button for actuation) or other device. “Axial,” as used herein, refers to the longitudinal direction of the of the delivery pen or other device, and “radial,” as used herein, refers to the direction perpendicular to the axial direction.

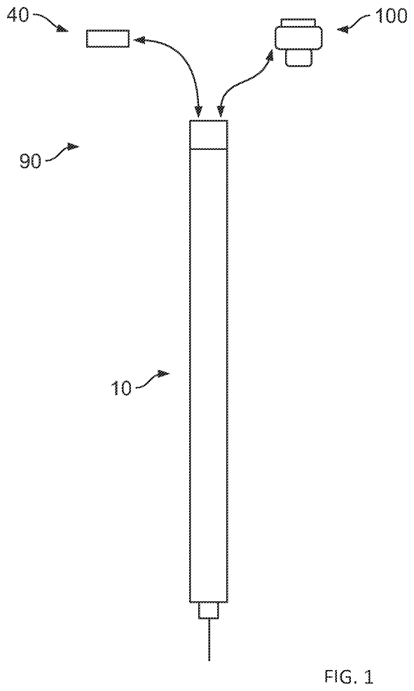

Embodiments described herein relate to dose delivery pen systems and methods of using the same. Referring to FIG. 1, a delivery pen system 90 disclosed herein generally includes a delivery pen 10, a button 40 selectively couplable to the delivery pen 10, and a dosing module 100 selectively couplable to the delivery pen 10. In some embodiments, coupling the button 40 to the delivery pen 10 can allow for a standard administration of a dose of medication through the delivery pen 10 by actuating the button 40. In some embodiments, coupling the dosing module 100 to the delivery pen 10 can allow for measuring one or more parameters of an administration of a dose of medication, such as the size of the dose administered, while the dose of medication is administered through the delivery pen 10. In some embodiments, the medication can be administered by actuating the dosing module 100 coupled to the delivery pen 10. In some embodiments, the delivery pen 10 and the button 40 can be disposable. In some embodiments, the dosing module 100 can be re-usable. In some embodiments, the dosing module 100 can be used with a plurality of delivery pens 10 throughout the lifetime of the dosing module 100.

In some embodiments, the dosing module 100 and the delivery pen 10 can be specifically designed for use with each other. That is, in some embodiments, the dosing module 100 can include one or more portions for tightly inserting into and coupling with the delivery pen 10. This can increase the reproducibility of attaching the dosing module 100 to a delivery pen 10, reduce the variability in measuring an administered dose with the dosing module 100, and reduce the footprint of the delivery pen 10 and dosing module 100 assembly. In some embodiments, the dosing module 100 can include one or more portions that are continuous with an outer surface of the delivery pen 10 to reduce the footprint of the delivery pen 10 and dosing module 100 assembly and increase ergonomics of the assembly. This offers a notable improvement over current modular dose measuring devices, which are commonly one-size-fits-all. Therefore, current modular dose measuring devices sized to fit onto an exterior of a plurality of delivery pen designs often lack in ergonomics and unnecessarily increase the footprint of the assemblies. Moreover, such current modular dose measuring devices do not securely couple to many delivery pens, resulting in an undesirable amount of “play” within the system. This can be exhibited by a lack of reproducibility and precision in the coupling of current modular dose measuring devices with delivery pens, which in turn limits the accuracy of measuring a size of an administered dose. An amount of play, as described herein, may generally refer to a variability in positioning of one or more components of a dose measuring device and/or a delivery pen. An amount of play, as described herein, may generally refer to an unintended amount of movement of two or more components of an assembly, including a dose measuring device and a delivery pen, relative each other. The greater the amount of play, the greater variability or amount of movement between the two components.

In some embodiments described herein, the dosing module 100 can include one or more sensing devices, such as electromechanical encoder wheels, that directly couple to the drive assembly of the delivery pen 10. This can increase the accuracy with which the one or more sensing devices measure the size of a dose of medication administered by the drive assembly of the delivery pen 10.

### Delivery Pen Drive Mechanisms

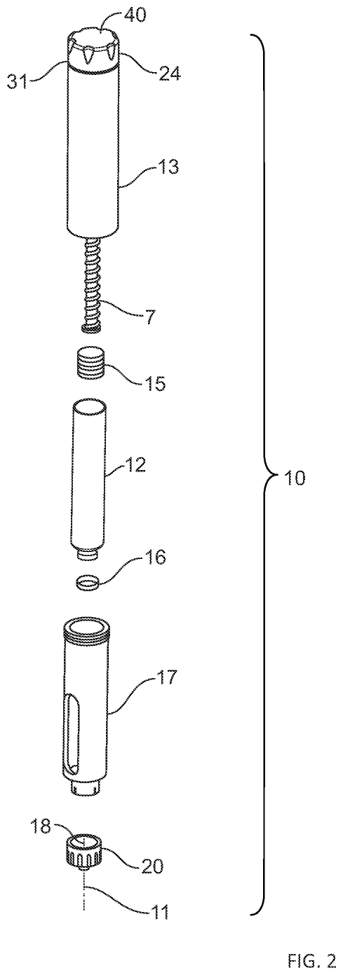

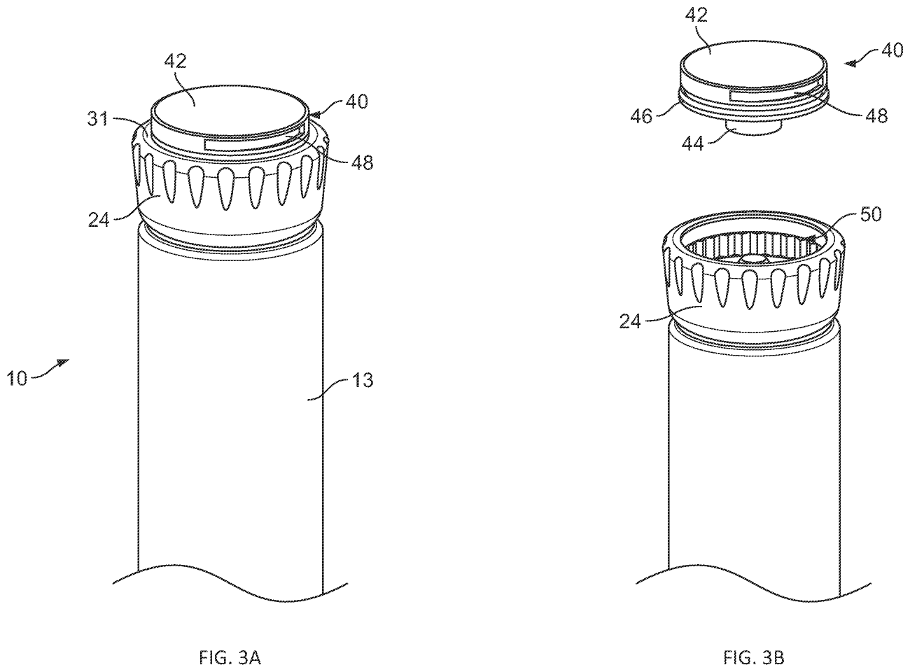

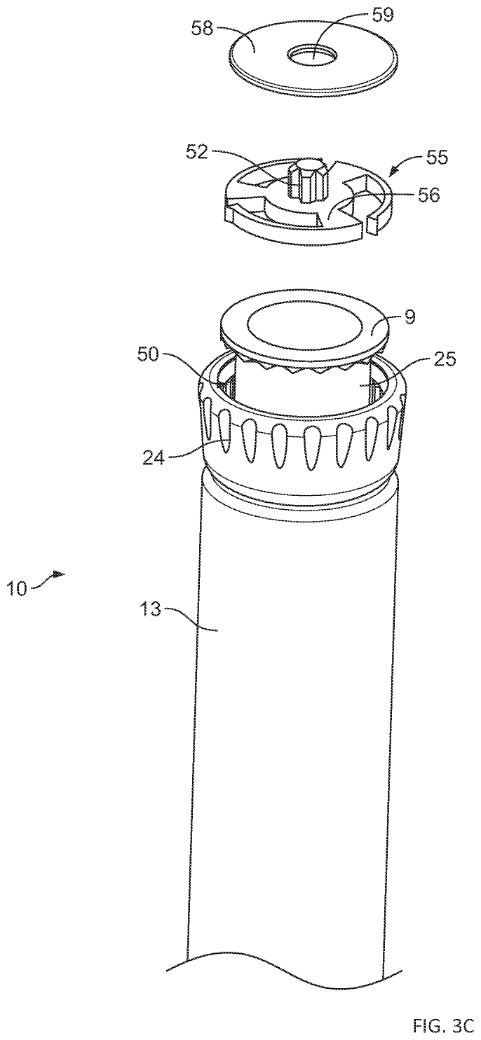

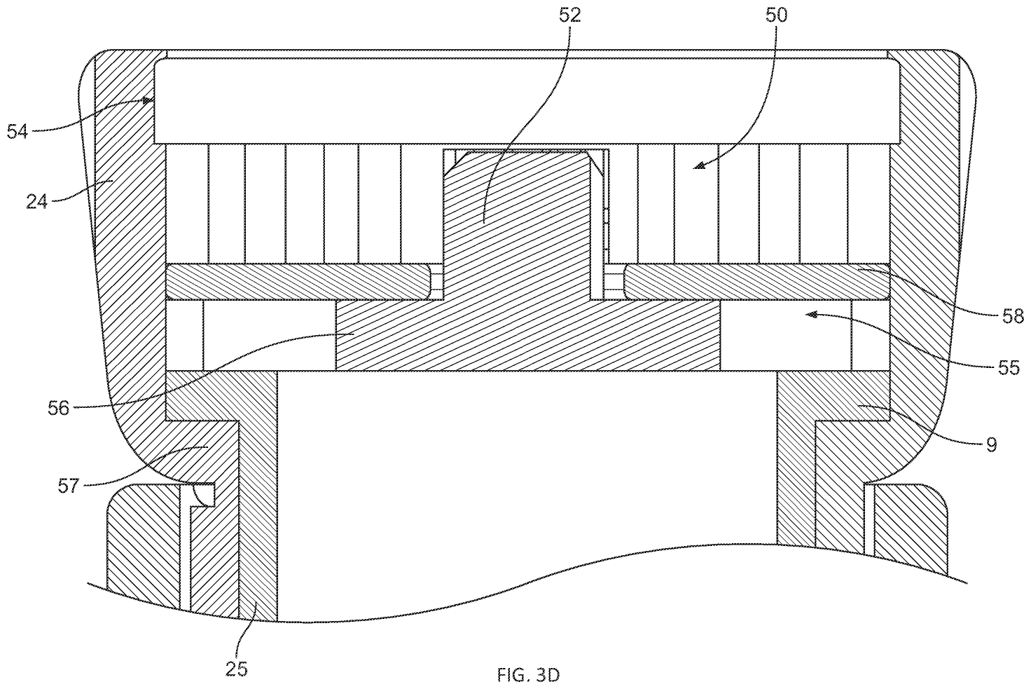

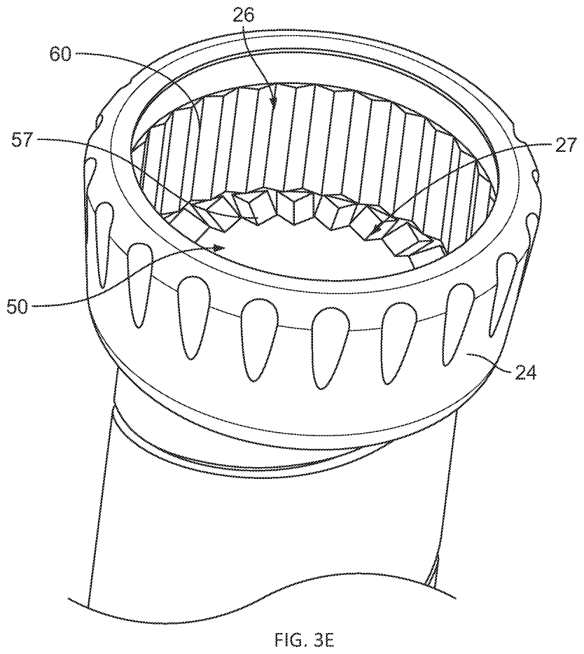

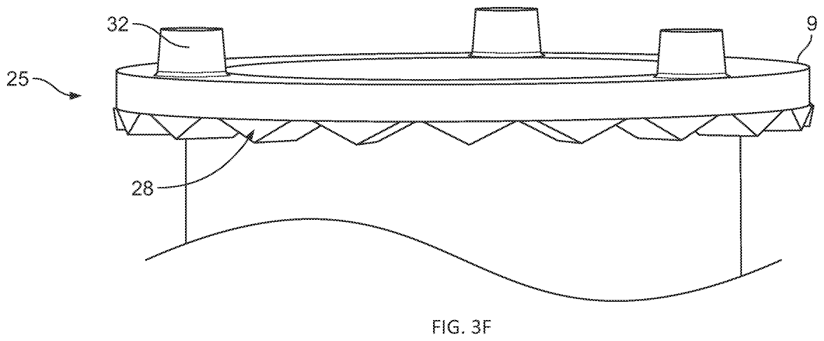

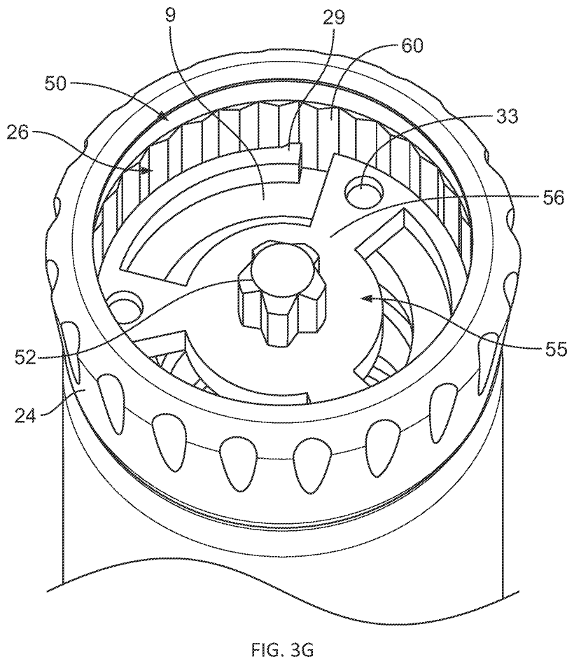

Referring now to FIG. 2, an exploded view of an exemplary delivery pen 10 is depicted. In some embodiments, the delivery pen 10 can be disposable. In some embodiments, the delivery pen 10 can include an outer sleeve 13. In some embodiments, the outer sleeve 13 can contain at least some of the driving mechanisms of the delivery pen 10 within. In some embodiments, the outer sleeve 13 contains an inner sleeve 25 (FIG. 3C) within. In some embodiments, the inner sleeve 25 (FIG. 3C) can be part of the driving mechanism of the delivery pen 10. In some embodiments, the outer sleeve 13 can contain medication supply to be administered to a patient. In some embodiments, the outer sleeve 13 can provide a gripping surface for a user to grip when administering medication to a patient. In some embodiments, the delivery pen 10 includes a dose knob 24 positioned at a proximal end 31 of the outer sleeve 13. The dose knob 24 can be rotatable with respect to the outer sleeve 13 of the delivery pen 10. In some embodiments, the user can rotate the dose knob 24 to selectively set a desired volume of a dose of medication to be injected to a patient. In some embodiments, rotation of the dose knob 24 in a first direction can axially translate the dose knob 24 away from the proximal end 31 of the outer sleeve 13, thereby increasing the volume of a dose of medication to be administered with the delivery pen 10. In some embodiments, rotation of the dose knob 24 in a second direction can axially translate the dose knob toward the proximal end 31 of the outer sleeve 13, thereby decreasing the volume of a dose of medication to be administered with the delivery pen 10. In some embodiments, a button 40 can be coupled to a proximal end of the dose knob 24. In some embodiments, the button 40 can be non-fixedly coupled to the proximal end of the dose knob 24. In some embodiments, the button 40 can be rotatably coupled to the dose knob 24 such that the dose knob 24 can rotate with respect to the button 40. In some embodiments, the button 40 and the dose knob 24 can be rotationally independent. In some embodiments, the button 40 can be non-rotatably coupled to the dose knob 24 such that the dose knob 24 and the button 40 rotate together. A user can apply an axial force to the button 40 to depress the button 40 and dose knob 24 axially toward the proximal end 31 of the outer sleeve 13, thereby activating the driving mechanism to administer a dose of medication from the delivery pen 10.

The driving and delivery mechanisms of the delivery pen 10 should be understood by a person having ordinary skill in the art and are, therefore, not discussed in detail herein. Generally, however, in some embodiments and by way of non-limiting example, depression of the button 40 and dose knob 24 in the distal direction injects the dosed medication via a lead screw 7 and a plunger 15 through a medicament cartridge 12, which is attached to the delivery pen 10 through a lower housing 17. In some embodiments, the distal movement of the plunger 15 within the medicament cartridge 12 causes medication to be forced into a needle 11 of a hub 20. In some embodiments, the medicament cartridge 12 can be sealed by a septum 16, which can be punctured by a septum penetrating needle or cannula 18 located within the hub 20. In some embodiments, the hub 20 can be screwed onto the lower housing 17, although other attachment means may be used. It should be appreciated that the foregoing description is merely one representative example of driving and delivery mechanisms of the delivery pen 10, and other designs for the driving and delivery mechanism of the delivery pen 10 are contemplated herein.

### Dummy Button

Referring now to FIGS. 3A and 3B, the dose knob 24 and button 40 will be discussed in greater detail. FIG. 3A depicts a perspective view of the dose knob 24 with the button 40 positioned at a proximal end thereof. FIG. 3B depicts an exploded view of the dose knob 24 and the button 40. As noted above, the button 40 can be removably or non-fixedly coupled to the dose knob 24. In some embodiments, the button 40 can include an actuation surface 42. The actuation surface 42 can extend proximally from the proximal end of the dose knob 24 when the button 40 is coupled to the dose knob 24 and provide a surface by which the user may actuate the button 40 and, therefore, the driving and delivery mechanism of the delivery pen 10. In some embodiments, the button 40 can include a stem 44 that sits within a well 50 of the dose knob 24 when the button 40 is coupled to the dose knob 24. In some embodiments, the stem 44 can removably couple to or otherwise engage one or more surfaces of the dose knob 24, such as a spline shaft 52 (FIGS. 3C and 3D), or one or more surfaces of the driving and delivery mechanisms, such as the inner sleeve 25 (FIGS. 3C and 3D), to impart an activation force onto the driving and delivery mechanisms of the delivery pen 10 when the button 40 is depressed. In some embodiments, the button 40 can include a flange 46 sized and shaped to engage a groove 54 (FIG. 3D) of the dose knob 24. For instance, in some embodiments, the flange 46 can form a snap fit with the groove 54 (FIG. 3D) of the dose knob 24 to couple the button 40 with the dose knob 24.

In some embodiments, the button 40 can include an access slot 48. In some embodiments, the access slot 48 can be formed in a peripheral edge of the button 40. The access slot 48 can be particularly formed in a surface of the button 40 that is exposed from the dose knob 24 when the button 40 is coupled to the dose knob 24. In some embodiments, the access slot 48 can generally be an indentation, partial opening, or opening in a surface of the button 40. The access slot 48 can generally provide a grip for the user to easily provide a force to the button 40. For instance, the access slot 48 can provide a grip for the user to more easily provide a force to the button 40 in the proximal direction. In some embodiments, providing a force to the button 40 in the proximal direction can pull, push, or pry the button 40 from the dose knob 24 such that the button 40 can be removed from the dose knob 24 and delivery pen 10. In other words, the force in the proximal direction applied to the button 40 can overcome the retention forces coupling the button 40 to the dose knob 24 to remove the button 40 from the dose knob 24. In some embodiments, the force in the proximal direction applied to the button 40 can overcome the retention forces exhibited on the flange 46 by the groove 54 (FIG. 3D) to remove the button 40 from the well 50 of the dose knob 24, as depicted in FIG. 3B.

While the access slot 48 has been discussed herein as providing a grip for a user to remove the button 40 from the dose knob 24, it should be appreciated that one or more other indentations or protrusions can be formed in the surface of the button 40 to provide a grip for the user to remove the button 40 from the dose knob 24. In some embodiments, the button 40 can include an internal, compliant spring mechanism and be deformable. That is, a user can provide a force to the button 40, such as providing a radially inward squeezing force to the button 40, to reduce the diameter of the button 40. Reduction of the diameter of the button 40 can move the flange 46 out of contact with the groove 54 (FIG. 3D), thereby allowing the user to remove the button 40 from the well 50 of the dose knob 24. The button 40 can return to its original or relaxed diameter when the squeezing force is removed from the button 40.

### Internal Components of Dose Knob

Referring now to FIGS. 3C-3G the dose knob 24 will be discussed in greater detail. FIG. 3C depicts an exploded view of the dose knob 24 with the button 40 (FIGS. 3A and 3B) removed from the dose knob 24. FIG. 3D depicts a cross-sectional view of the dose knob 24 with the button 40 (FIGS. 3A and 3B) removed from the dose knob 24. FIG. 3E depicts a perspective view of the dose knob 24 with the well 50 of the dose knob 24 empty. FIG. 3F depicts a perspective view of a proximal end of the inner sleeve 25. FIG. 3G depicts a perspective view of the dose knob 24. In some embodiments, the inner sleeve 25 is at least partially received in the well 50 of the dose knob 24. For instance, in some embodiments, the inner sleeve 25 can include a flange 9 that sits on a lower lip 57 of the dose knob 24. In some embodiments, the lower lip 57 can define a distal end of the well 50 of the dose knob 24. In some embodiments, a shaft spring 55 is positioned in the well 50 of the dose knob 24. The shaft spring 55 can generally include a base 56 and a spline shaft 52 extending proximally from the base 56. In some embodiments, the shaft spring 55 is positioned adjacent to a proximal end of the inner sleeve 25. Particularly, in some embodiments, a distal surface of the base 56 of the shaft spring 55 is positioned adjacent a proximal surface of the flange 9 of the inner sleeve 25. In some embodiments, a disk 58 can be positioned within the well 50 of the dose knob 24. In some embodiments, the disk 58 is positioned adjacent a proximal surface of the base 56 of the shaft spring 55. Particularly, in some embodiments, a distal surface of the disk 58 is positioned adjacent a proximal surface of the base 56 of the shaft spring 55. In some embodiments, the disk 58 includes an annular recess 59 sized to receive the spline shaft 52 of the shaft spring 55 therethrough.

In some embodiments, one or more internal surfaces of the well 50 of the dose knob 24, one or more surfaces of the inner sleeve 25, and/or one or more surfaces of the shaft spring 55 can include corresponding features such that the dose knob 24, inner sleeve 25, and/or shaft spring 55 can couple to rotate together. In some embodiments the dose knob 24 and the inner sleeve 25 can include corresponding features that allow for the dose knob 24 and the inner sleeve 25 to rotate together. For instance, in some embodiments, the lower lip 57 of the dose knob 24 and a distal surface of the flange 9 can include corresponding ribbing, such as the ribbing 27 of the lower lip 57 and the ribbing 28 of the flange 9, that mate together to allow the dose knob 24 and inner sleeve 25 to rotate together. In some embodiments, the shaft spring 55 and the dose knob 24 can include corresponding features that allow for the dose knob 24 and the shaft spring 55 to rotate together. In some embodiments, a peripheral surface of the base 56 of the shaft spring 55 and a radially internal surface of the well 50 of the dose knob 24 can include corresponding ribbing, such as the ribbing 26 of a radially inner surface of a wall 60 of the well 50 and the rib 29 of the base 56 of the shaft spring 55, that mate together to allow the dose knob 24 and shaft spring 55 to rotate together. In some embodiments, the shaft spring 55 and the inner sleeve 25 can include corresponding features that allow for the shaft spring 55 and the inner sleeve 25 to rotate together. In some embodiments, a distal surface of the base 56 of the shaft spring 55 and a proximal surface of the flange 9 of the inner sleeve 25 can include corresponding features that mate together to allow the inner sleeve 25 and shaft spring 55 to rotate together. For instance, in some embodiments, the flange 9 includes pegs 32 extending from a proximal surface of the flange 9, and the base 56 of the shaft spring 55 includes bores 33 sized to receive the pegs 32.

While corresponding ribbing and peg/bore interactions have been described herein as allowing a non-rotatable coupling between the dose knob 24, shaft spring 55, and/or inner sleeve 25, such that the dose knob 24, shaft spring 55, and/or inner sleeve 25 do not rotate with respect to each other, it should be appreciated that the dose knob 24, shaft spring 55, and inner sleeve 25 can respectively include any geometric features that can mate together to allow for a non-rotatable coupling. In some embodiments, the corresponding features of the dose knob 24, shaft spring 55, and/or inner sleeve 25 can non-rotatably couple the dose knob 24, shaft spring 55, and/or inner sleeve 25 only when an injection is taking place. For instance, in some embodiments, the corresponding features of the dose knob 24, shaft spring 55, and/or inner sleeve 25 can non-rotatably couple the dose knob 24, shaft spring 55, and/or inner sleeve 25 only when axial force in the distal direction is applied to the shaft spring 55, inner sleeve 25, and/or dose knob 24 (to force the corresponding features into contact with each other, for instance). Additionally, it should be appreciated that indirect, non-rotatable couplings can be formed between the dose knob 24, shaft spring 55, and/or inner sleeve 25. For instance, the dose knob 24 can be non-rotatably coupled to the shaft spring 55, and the inner sleeve 25 can be non-rotatably coupled to the shaft spring 55, as described above. In some embodiments, the dose knob 24 and the inner sleeve 25 may not share corresponding features such that the dose knob 24 and inner sleeve 25 are directly non-rotatably coupled. However, the dose knob 24 and the inner sleeve 25 can be non-rotatably coupled to each other by way of both of the dose knob 24 and the inner sleeve 25 being non-rotatably coupled to the shaft spring 55.

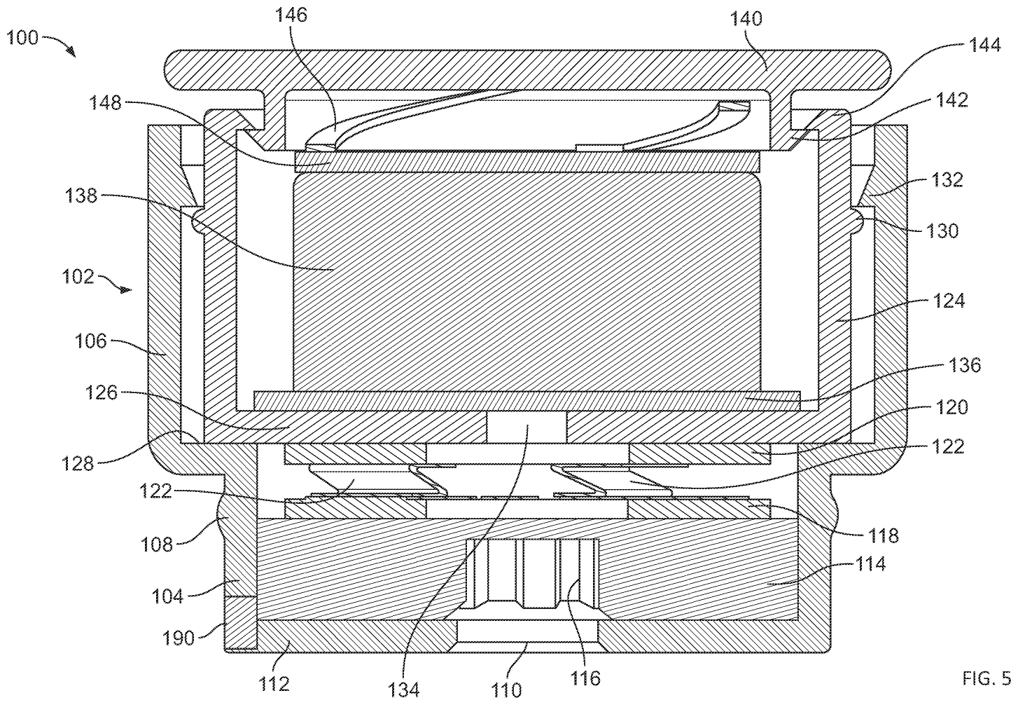

While embodiments have been described herein including the shaft spring 55, it should be appreciated that this is a non-limiting example. For instance, in some embodiments, the dose knob 24 can include any suitable component with a shaft (e.g. the spline shaft 52) for coupling to the spline hole plate 114 (FIG. 5) within the well 50 of the dose knob 24, as will be discussed in detail below. In some embodiments, the dose knob 24 can include any suitable component with a surface (e.g. the base 56 or a wall of the well 50) for coupling to a member of the driving mechanism of the delivery pen 10. Similarly, while embodiments have been described herein with the inner sleeve 25 at least partially received within the well 50 of the dose knob 24, it should be appreciated that this is a non-limiting example. For instance, in some embodiments, the dose knob 24 can include any desirable component of the driving mechanism of the delivery pen 10 within the well 50 for coupling with a wall of the well 50 and/or the shaft spring 55 (or other suitable component within the dose knob 24).

### Dosing Module

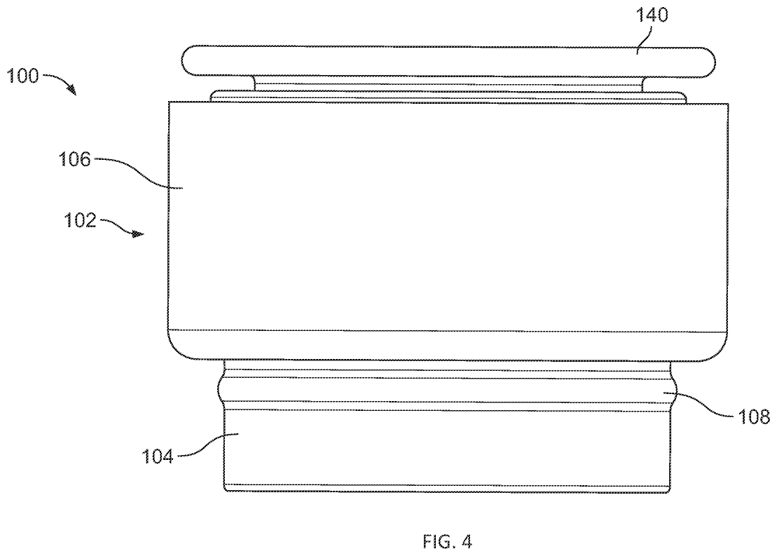

Referring now to FIG. 4, a side view of a dosing module 100 is depicted. In some embodiments, the dosing module 100 can be selectively coupled to the delivery pen 10. In some embodiments, the dosing module 100 can be re-usable. That is, in some embodiments, the delivery pen 10 can be disposed of following an administration of medication to a patient, or after a set period of use, such as one week, and the dosing module 100 can be used with multiple delivery pens 10 throughout the operational lifetime of the dosing module 100. In some embodiments, the dosing module 100 generally includes an outer shell 102. In some embodiments, the outer shell 102 can be defined by a stem 104 and a body 106. In some embodiments, the stem 104 extends distally from a distal end of the body 106. In some embodiments, the stem 104 can have a smaller diameter or width than the body 106. In some embodiments, the stem 104 can include a retaining ridge 108 extending radially outward from a peripheral surface of the stem 104. In some embodiments, the dosing module 100 includes a button 140 extending from a proximal end of the body 106 of the outer shell 102.

### Internal Components of Dosing Module

Referring now to FIG. 5, a cross-sectional view of the dosing module 100 is depicted. In some embodiments, the dosing module 100 includes an opening 110 through a distal wall 112 of the stem 104. In some embodiments, the opening 110 can be chamfered. In some embodiments, the opening 110 can be beveled.

In some embodiments, the dosing module 100 includes a spline hole plate 114. In some embodiments, the spline hole plate 114 is positioned adjacent the distal wall 112 of the stem 104 of the outer shell 102. In some embodiments, a distal surface of the spline hole plate 114 is positioned adjacent a proximal surface of the distal wall 112 of the stem 104 of the outer shell 102. In some embodiments, the distal surface of the spline hole plate 114 is rigidly coupled to the proximal surface of the distal wall 112 of the stem 104 of the outer shell 102, such that the outer shell 102 and the spline hole plate 114 are rotatable together. In some embodiments, the distal surface of the spline hole plate 114 is rigidly coupled to the proximal surface of the distal wall 112 of the stem 104 of the outer shell 102 through adhesive, welding, press fit, or other suitable means. In some embodiments, the spline hole plate 114 includes a spline hole 116 extending at least partially therethrough. In some embodiments, the spline hole 116 can extend through a distal surface of the spline hole plate 114. In some embodiments, the spline hole 116 can be chamfered. In some embodiments, the spline hole 116 can be beveled. In some embodiments, the spline hole 116 of the spline hole plate 114 is positioned coaxially with the opening 110 of the distal wall 112 of the stem 104. In some embodiments, the spline hole 116 includes one or more grooves or surface features within or along the interior surface of the spline hole 116 to mate with the spline shaft 52 (FIGS. 3C and 3D) of the shaft spring 55 (FIGS. 3C and 3D).

In some embodiments, the dosing module 100 includes an encoder wheel 118. In some embodiments, the encoder wheel 118 is coupled to the spline hole plate 114 such that the encoder wheel 118 and the spline hole plate 114 are rotatable together. In some embodiments, a distal surface of the encoder wheel 118 is rigidly coupled to a proximal surface of the spline hole plate 114, such that the encoder wheel 118 and the spline hole plate 114 are rotatable together. In other words, in some embodiments, the encoder wheel 118 and the spline hole plate 114 are rigidly coupled such that rotation of the spline hole plate 114 results in rotation of the encoder wheel 118. In some embodiments, the distal surface of the encoder wheel 118 is rigidly coupled to the proximal surface of the spline hole plate 114 through adhesive, welding, press fit, or other suitable means. In some embodiments, the encoder wheel 118 includes one or more electrically conductive pads printed or etched onto a printed circuit board (PCB) or other suitable material.

In some embodiments, the dosing module 100 includes a secondary conductive plate 120. The secondary conductive plate 120 can be a PCB or other suitable material. In some embodiments, the secondary conductive plate 120 can be fixedly coupled to one or more contact clips 122. In some embodiments, the one or more contact clips 122 can be electrically conductive. In some embodiments, the secondary conductive plate 120 is communicatively and electrically coupled to the one or more contact clips 122 through conductive adhesive, soldering, or the like. In some embodiments, distal ends of the contact clips 122 can contact the proximal surface of the encoder wheel 118. In some embodiments, the contact clips 122 can be maintained in contact with the proximal surface of the encoder wheel 118 by means of compression from the components of the dosing module 100 positioned proximally of the contact clips 122. In some embodiments, the distal ends of the contact clips 122 are not coupled to the encoder wheel 118. In some embodiments, the encoder wheel 118 can freely rotate with respect to the contact clips 122. The contact clips 122 can transmit an electrical signal to the secondary conductive plate 120 based on the degree of rotation of the encoder wheel 118. While contact clips 122 are discussed in detail herein, it should be appreciated that the dosing module 100 can include any suitable sensing means that can detect rotation of the encoder wheel and generate or transmit a signal based on the rotation.

In some embodiments, the dosing module 100 includes an inner shell 124. In some embodiments, the inner shell 124 can be predominantly positioned within the body 106 of the outer shell 102. In some embodiments, a distal surface of a distal wall 126 of the inner shell 124 can be positioned adjacent a lip 128 of the body 106 of the outer shell 102. In some embodiments, interaction between the distal surface of the distal wall 126 and the lip 128 of the body 106 of the outer shell 102 can at least partially maintain the inner shell 124 predominantly within the body 106 of the outer shell 102. In some embodiments, the inner shell 124 includes a ridge 130 projecting radially outward from a peripheral surface of the inner shell 124, and the body 106 of the outer shell 102 includes a corresponding protrusion 132 projecting radially inward from an inner surface of the body 106 of the outer shell 102. In some embodiments, the ridge 130 and the protrusion 132 form a snap fit to maintain the inner shell 124 predominantly within the body 106 of the outer shell 102. In some embodiments, the outer shell 102 and the inner shell 124 are not rigidly coupled, such that the outer shell 102 can freely rotate with respect to the inner shell 124. In some embodiments, the inner shell 124 can be coupled to the secondary conductive plate 120. In some embodiments, a distal surface of the distal wall 126 of the inner shell 124 can be rigidly coupled to a proximal surface of the secondary conductive plate 120. In some embodiments, the distal surface of the distal wall 126 of the inner shell 124 is rigidly coupled to the proximal surface of the secondary conductive plate 120 through adhesive, welding, press fit, or other suitable means. In some embodiments, an opening 134 can extend through the distal wall 126 of the inner shell 124.

In some embodiments, the dosing module 100 includes a primary conductive plate 136. The primary conductive plate 136 can be a PCB or other suitable material. In some embodiments, the primary conductive plate 136 can be coupled to the inner shell 124. In some embodiments, a distal surface of the primary conductive plate 136 can be rigidly coupled to a proximal surface of the distal wall 126 of the inner shell 124. In some embodiments, the distal surface of the primary conductive plate 136 can be rigidly coupled to the proximal surface of the distal wall 126 of the inner shell 124 through adhesive, welding, press fit, or other suitable means. In some embodiments, the primary conductive plate 136 is communicatively and electrically coupled to the secondary conductive plate 120, such that one or more signals may be communicated between the two. For instance, in some embodiments, one or more electrically conductive wires can couple the primary conductive plate 136 with the secondary conductive plate 120. In some embodiments, the one or more electrically conductive wires coupling the primary conductive plate 136 with the secondary conductive plate 120 can extend from the secondary conductive plate 120 to the primary conductive plate 136 through the opening 134 in the distal wall 126 of the inner shell 124.

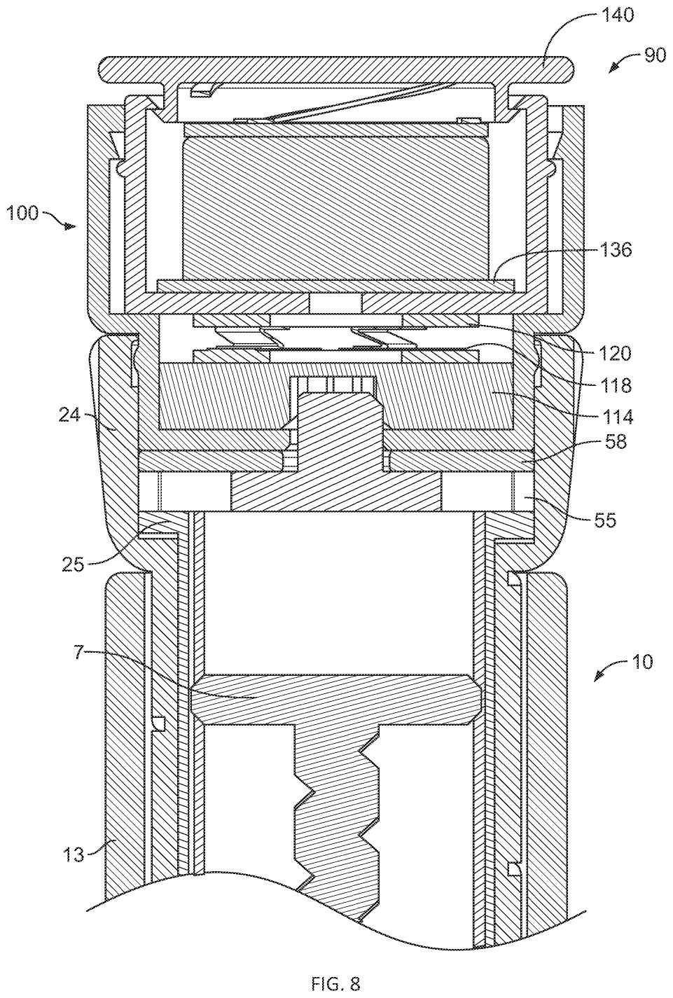

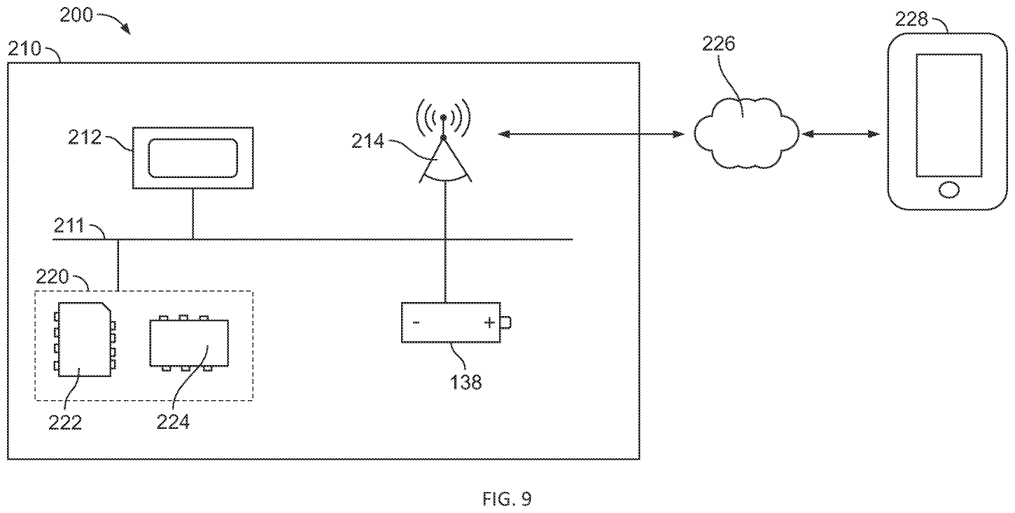

In some embodiments, one or more electronic components or modules can be coupled to the primary conductive plate 136. In some embodiments, the one or more electronic components can be soldered to the primary conductive plate 136. In some embodiments, the one or more electronic components can be electrically and communicatively coupled with each other through the primary conductive plate 136. In some embodiments, the one or more electronic components can be electrically and communicatively coupled with the secondary conductive plate 120, a circuit board 148, and other internal components of the dosing module 100 electrically coupled to the primary conductive plate 136. In some embodiments, the one or more electronic components can include a battery 138, a controller 220 (FIG. 9), a network interface hardware 214 (FIG. 9), and/or other electronic modules. In some embodiments, the battery 138 can be a coin cell battery. In some embodiments, the battery 138 is specified such that it can provide power to run the systems of the dosing module 100 for a determined lifetime of the dosing module 100.

In some embodiments, the dosing module 100 includes a button 140. In some embodiments, the button 140 can extend proximally from a proximal end of the inner shell 124. In some embodiments, the button 140 can extend proximally from the proximal end of the body 106 of the outer shell 102. In some embodiments, the button 140 includes hooks 142 extending distally from the button 140. In some embodiments, the hooks 142 can engage a lip 144 of the inner shell 124. In some embodiments, engagement of the hooks 142 with the lip 144 of the inner shell 124 can maintain the button 140 coupled to the inner shell 124 of the dosing module 100. In some embodiments, the button 140 is depressible in an axial direction to impart an activation force onto the driving and delivery mechanisms of the delivery pen 10 (FIG. 1), and thereby administer a dose of medication to a patient.

In some embodiments, the dosing module 100 can include conductive spring clips 146. In some embodiments, proximal ends of the conductive spring clips 146 can be coupled to a distal surface of the button 140. In some embodiments, the proximal ends of the conductive spring clips 146 can be in contact with the distal surface of the button 140 when the button 140 and the conductive spring clips 146 are in a relaxed state. In some embodiments, the distal surface of the button 140 is brought into contact with the proximal ends of the conductive spring clips 146 when the button 140 is depressed distally. The distal ends of the conductive spring clips 146 can be coupled to the circuit board 148. In some embodiments, the circuit board 148 can be a PCB. In some embodiments, depression of the button 140 distally causes the proximal ends of the conductive spring clips 146 to contact each other and/or the circuit board 148. In some embodiments, when the proximal ends of the conductive spring clips 146 contact each other and/or the circuit board 148, a circuit is completed, generating an electrical signal. In some embodiments, the electrical signal can be indicative of a user depressing the button 140 to begin administration of a dose of medication. In some embodiments, the circuit board 148 can be communicatively and electrically coupled to the primary conductive plate 136, by means of one or more conducting wires, for instance, to transmit the electrical signal to one or more electronic modules coupled to the primary conductive plate 136. In some embodiments, the circuit board 148 can be coupled to the battery 138. In some embodiments, the distal surface of the circuit board 148 can be coupled to a proximal surface of the battery 138.

In some embodiments, the dosing module 100 can include a sensor 190. In some embodiments, the sensor 190 is coupled to or positioned on a surface of the stem 104 of the outer shell 102 of the dosing module 100. In some embodiments, the sensor 190 can be a switch. In some embodiments, the sensor 190 can be a capacitive sensor. In some embodiments, the sensor 190 can be configured to detect when the dosing module 100 is inserted into and/or removed from the dose knob 24 (FIG. 3D). In some embodiments, the sensor 190 can be configured to detect when the stem 104 is inserted into and/or removed from the well 50. The sensor 190 can be communicatively and electrically coupled to the primary conductive plate 136 and/or one or more electronic modules coupled thereto.

### Mechanical Connections Between Dose Knob and Dosing Module

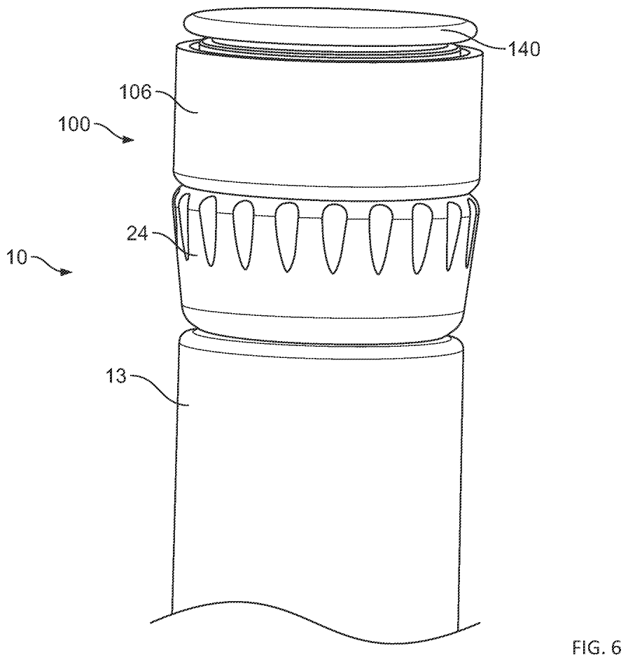

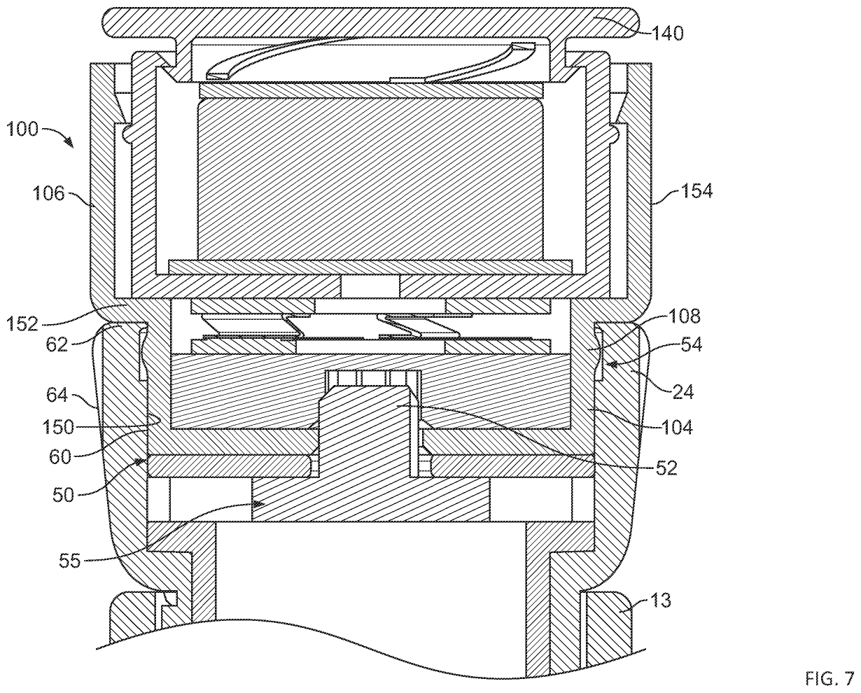

Referring now to FIGS. 5-7, attachments between the dosing module 100 and the delivery pen 10 will be discussed. FIG. 6 generally depicts a perspective view of the dosing module 100 coupled to the delivery pen 10. FIG. 7 generally depicts a cross-sectional view of the dosing module 100 coupled to the delivery pen 10. In some embodiments, the stem 104 of the outer shell 102 of the dosing module 100 is insertable into the well 50 of the dose knob 24. In some embodiments, when the stem 104 is inserted into the well 50 of the dose knob 24, the retaining ridge 108 of the dosing module 100 can engage, or be received in, the groove 54 of the dose knob 24. The retaining ridge 108 and the groove 54 can be sized to form a snap fit to secure the dosing module 100 to the dose knob 24. The retaining force exhibited on the retaining ridge 108 by the groove 54 can be overcome by a user applying sufficient force to the dosing module 100 in the proximal direction, for instance, to pull the stem 104 out of the well 50 of the dose knob 24 to selectively decouple the dosing module 100 from the delivery pen 10.

In some embodiments, the diameter or outer dimension of a peripheral edge 150 of the stem 104 of the outer shell 102 of the dosing module 100 can be sized such that the peripheral edge 150 of the stem 104 contacts or is adjacent to the radially inner surface of the wall 60 of the well 50 of the dose knob 24. The stem 104 and the dose knob 24 being so particularly sized can reduce undesired movement between the dosing module 100 and delivery pen 10 in the assembly such that variability and error in dose measurements can be reduced. In some embodiments, the peripheral edge 150 of the stem 104 of the outer shell 102 of the dosing module 100 and the radially inner surface of the wall 60 of the well 50 of the dose knob 24 can form a friction fit with each other. The stem 104 of the outer shell 102 of the dosing module 100 being received within the well 50 of the dose knob 24 can reduce the footprint of the assembly of the dosing module 100 and delivery pen 10 compared to current devices, thereby increasing user ergonomics and comfort during operation of the delivery pen 10.

In some embodiments, the body 106 of the outer shell 102 of the dosing module 100 can have a greater diameter or outer dimension than the stem 104 of the outer shell 102. In some embodiments, the body 106 of the outer shell 102 is sized such that when the stem 104 is received in the well 50 of the dose knob 24, the body 106 of the outer shell 102 is positioned outside of the well 50 of the dose knob 24. In some embodiments, the body 106 of the outer shell 102 is sized such that when the stem 104 is received in the well 50 of the dose knob 24, the body 106 of the outer shell 102 is positioned distally of the dose knob 24. In some embodiments, the body 106 of the shell 102 is sized such that when the stem 104 is received in the well 50 of the dose knob 24, a distal surface of a distal end wall 152 of the body 106 of the outer shell 102 contacts a proximal surface of a proximal end wall 62 of the dose knob 24. In some embodiments, the diameter or outer dimension of a peripheral edge 154 of the body 106 of the outer shell 102 of the dosing module 100 can be sized to be substantially equal to the diameter or outer dimension of a peripheral edge 64 of the dose knob 24, such that the body 106 of the outer shell 102 of the dosing module 100 and the dose knob 24 form a substantially continuous outer surface. In some embodiments, the diameter or outer dimension of the peripheral edge 154 of the body 106 of the outer shell 102 of the dosing module 100 can be sized to be less than the diameter or outer dimension of the peripheral edge 64 of the dose knob 24. The substantially continuous outer surface or the outer dimension of the peripheral edge 154 of the body 106 being less than the outer dimension of the peripheral edge 64 of the dose knob 24 can reduce the footprint of the assembly of the dosing module 100 and delivery pen 10 compared to current devices, thereby increasing user ergonomics and comfort during operation of the delivery pen 10.

In some embodiments, when the stem 104 of the outer shell 102 of the dosing module 100 is received in the well 50 of the dose knob 24, the spline shaft 52 of the shaft spring 55 is received within the opening 110 through the distal wall 112 of the stem 104 of the outer shell 102. In some embodiments, the chamfered or beveled shape of the opening 110 can assist in insertion of the spline shaft 52 through the opening 110. In some embodiments, when the stem 104 of the outer shell 102 of the dosing module 100 is received in the well 50 of the dose knob 24, the spline shaft 52 of the shaft spring 55 is received within the spline hole 116 extending at least partially through the spline hole plate 114 and meshes with the spline hole 116. In some embodiments, the chamfered or beveled shape of the spline hole 116 can assist in insertion of the spline shaft 52 into the spline hole 116.

### Operation of the Dose Delivery

Operation of the delivery pen 10 and dosing module 100 assembly will now be discussed with reference to FIGS. 3D, 5, 7, and 8. FIG. 8 generally depicts a cross-sectional view of the delivery pen system 90 with the dosing module 100 coupled to the delivery pen 10. In such embodiments, an original dosing button (e.g., the button 40 depicted in FIG. 1) that initially was part of the delivery pen 10 is completely removed and replaced with the dosing module 100 of the present disclosure. With the dosing module 100 coupled to the dose knob 24 of the delivery pen 10 that has been altered to remove an original dosing button, a user can administer a dose of medication as desired. To begin administering a dose, the user depresses the button 140 of the dosing module 100 axially downward in the distal direction. In turn, the button 140 can depress the conductive spring clips 146, as discussed above, to complete a circuit therebetween. Following completion of the circuit, the circuit board 148 can then send an electrical signal to the controller 220 (FIG. 9) coupled to the primary conductive plate 136, indicating the controller 220 (FIG. 9) should begin assessing the amount of medication administered or the size of the dose administered. In some embodiments, the controller 220 (FIG. 9) can assess the size of the dose for a set period of time after the button 140 is depressed, or after the circuit is completed between the conductive spring clips 146. In some embodiments, the controller 220 (FIG. 9) can assess the size of the dose for as long as the button 140 is depressed, or for as long as the circuit is completed between the conductive spring clips 146.

Axial depression of the button 140 in the distal direction, also, imparts an activation force onto the driving and delivery mechanisms of the delivery pen 10. For instance, in some embodiments, axial depression of the button 140 in the distal direction drives or rotates the inner sleeve 25 of the delivery pen 10, which can in turn rotate or drive the lead screw 7 or other components of the driving and delivery mechanism of the delivery pen 10. More specifically, in some embodiments, depression of the button 140 can cause the dosing module 100 to move distally, thereby applying a force to the disk 58, the shaft spring 55, and the inner sleeve 25 in the well 50 of the dose knob 24. In some embodiments, the axial force applied to the shaft spring 55 and the inner sleeve 25 can result in the corresponding features of the shaft spring 55, inner sleeve 25, and/or one or more inner surfaces of the well 50 (discussed with respect to FIGS. 3C-3G) mating such that the dose knob 24, one or more surfaces of the inner sleeve 25, and/or one or more surfaces of the shaft spring 55 couple to rotate together. In some embodiments, the axial force applied to the shaft spring 55 can result in the corresponding features of the shaft spring 55 and the inner sleeve 25 mating such that the shaft spring 55 and the inner sleeve 25 couple to rotate together. In some embodiments, the axial force applied to the inner sleeve 25 can result in the corresponding features of the inner sleeve 25 and the one or more inner surfaces of the well 50 mating such that the inner sleeve 25 and the dose knob 24 couple to rotate together. In some embodiments, the axial depression of the button 140 can impart an axial force to the dose knob 24 that moves the dose knob 24 distally. In some embodiments, the dose knob 24 can rotate as it translates due to a helical pattern on its exterior. In some embodiments, because the inner sleeve 25 is coupled to the dose knob 24, the inner sleeve 25 rotates along with the dose knob 24 during the injection process. The rotating inner sleeve 25 in turn rotates both the lead screw 7 and the shaft spring 55.

It should be understood that the degree of rotation of the inner sleeve 25 is indicative of the size of the dose of medication administered, with a greater degree of rotation correlating to a larger dose of medication administered. Rotation of the inner sleeve 25 can, in turn, result in rotation of the shaft spring 55 which is coupled to the inner sleeve 25, as discussed above. Rotation of the shaft spring 55 can, in turn, result in rotation of the spline hole plate 114 through the mating of the spline shaft 52 with the spline hole 116, as discussed above. Rotation of the spline hole plate 114 can, in turn, result in rotation of the encoder wheel 118 fixedly coupled to the spline hole plate 114. Therefore, during administration of a dose of medication, the inner sleeve 25, shaft spring 55, spline hole plate 114, and encoder wheel 118 can unitarily rotate. In some embodiments, for instance when the spline hole plate 114 is fixedly coupled to the stem 104 of the outer shell 102 of the dosing module 100, the inner sleeve 25, shaft spring 55, spline hole plate 114, encoder wheel 118, and outer shell 102 of the dosing module 100 can unitarily rotate. In some embodiments, the button 140, inner shell 124, and components housed within the inner shell 124 can remain rotationally stationary as any or all of the inner sleeve 25, shaft spring 55, spline hole plate 114, encoder wheel 118, and outer shell 102 of the dosing module 100 rotate. The button 140 and inner shell 124, in being rotationally stationary, can offer the user increased ergonomic comfort and control of the delivery pen 10.

In some embodiments, as the encoder wheel 118 rotates, the electrically conductive pads of the encoder wheel 118 rotate in and out of contact with the contact clips 122. Depending on the degree of rotation of the encoder wheel 118, a different electrical signal can be communicated to the secondary conductive plate 120 by the contact clips 122. In some embodiments, the secondary conductive plate 120 can then pass the electrical signal gathered from the contact clips 122 to the primary conductive plate 136 and/or one or more electronic modules coupled to the primary conductive plate 136, such as the controller 220 (FIG. 9). In some embodiments, the one or more electronic modules coupled to the primary conductive plate 136 can then determine and/or communicate the size of the dose of medication administered to the patient.

It should be appreciated that prior to administering a dose of medication, the dose knob 24 can be freely rotated to select a dose size. In some embodiments, the one or more internal surfaces of the well 50 of the dose knob 24, one or more surfaces of the inner sleeve 25, and/or one or more surfaces of the shaft spring 55 couple to rotate together under an axial depression force from the button 140. Therefore, in some embodiments, prior to an injection (i.e., prior to an axial depression force being applied to the button 140), a user can rotate the dose knob 24 without simultaneously rotating the inner sleeve 25, or other components of the driving mechanism of the delivery pen 10, such that a dose is not inadvertently administered when rotating the dose knob 24 to select a dose size.

### Alternative Embodiments

While embodiments have been described above where the conductive spring clips 146 and circuit board 148 generate a signal indicative of the button 140 being depressed when a circuit is completed by the conductive spring clips 146, it should be appreciated that one or more other sensors can be used to generate a signal indicative of the button 140 being depressed. For instance, in some embodiments, a capacitive sensor can be positioned in or on the button 140. In some embodiments, the capacitive sensor can be communicatively and electrically coupled to the primary conductive plate 136 and/or the one or more electronic modules coupled to the primary conductive plate 136. The capacitive sensor can detect the presence of a finger of a user on the button 140 and send a signal to the controller 220 (FIG. 9), indicating the controller 220 (FIG. 9) should begin assessing the amount of medication administered or the size of the dose administered. Similarly, in some embodiments, a magnetic sensor or a microswitch can be positioned in, on, or adjacent to the button 140 to generate a signal when a finger of a user is on the button 140 and/or when the button 140 is axially depressed in the distal direction.

While embodiments have been described above where the encoder wheel 118 is an electromechanical encoder, it should be appreciated that one or more other encoder types can be included in the dosing module 100 for generating a signal indicative of the size of the dose of medication administered. For instance, in some embodiments a magnetic encoder wheel 118 can be coupled to the spline hole plate 114, as discussed above. In some embodiments, the magnetic encoder wheel 118 can include a series of magnetic poles around the circumference of the magnetic encoder wheel 118, or the magnetic encoder wheel 118 can include a permanent magnet. In place of or in addition to the contact clips 122, a magnetic sensor can be positioned in the dosing module 100 to generate a signal based on a detected degree of rotation of the magnetic encoder wheel 118. In some embodiments, an optical encoder wheel 118 can be coupled to the spline hole plate 114, as discussed above. In some embodiments, the optical encoder wheel 118 can be made of a transparent material and include a series of opaque markings thereon. A light source, such as an LED light can be positioned within the dosing module 100 to emit a light to the optical encoder wheel 118. In place of the contact clips 122, a photodetector assembly or other sensor can be positioned opposite the light source to generate a signal based on the degree of rotation of the optical encoder wheel 118.

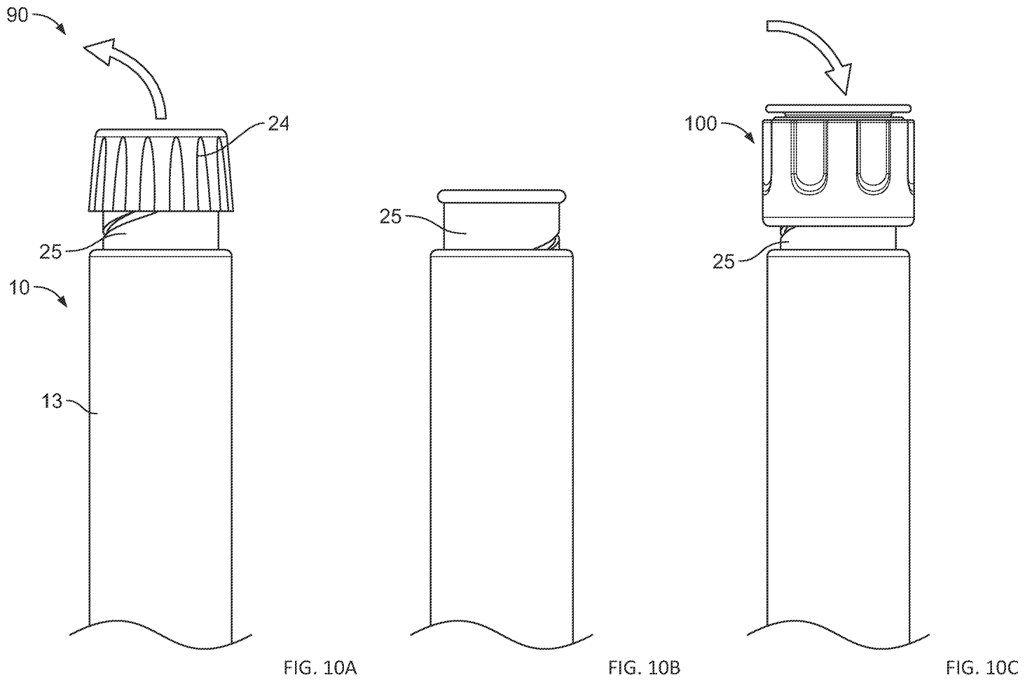

While embodiments have been described herein where the dosing module 100 is attached to the dose knob 24, it should be appreciated that other embodiments are contemplated. For instance, and referring now to FIGS. 10A-10C, an embodiment of the delivery pen system 90 is depicted. In some embodiments, as depicted, the dose knob 24 can be removed from the delivery pen 10 and the dosing module 100 can be attached directly to one or more components of the driving mechanism of the delivery pen 10, such as the inner sleeve 25.

### Electronic Control System

Referring now to FIG. 9, an operations system 200 can be provided for operating the dosing module 100, in particular, for determining a size of a dose of medication administered to a patient, for example, based on sensor measurements of the rotation of the encoder wheel 118. The operations system 200 can include a controller 220, the battery 138, or power supply, a display device 212, a network interface hardware 214, and a communication path 211 communicatively coupling these components, some or all of which may be disposed in an onboard control unit 210. Furthermore, the operations system 200 can be communicatively coupled to the circuit board 148 and one or more other sensors, such as the sensor 190, of the dosing module 100.

The controller 220 can include a processor 222 and a non-transitory electronic memory 224 to which various components are communicatively coupled. In some embodiments, the processor 222 and the non-transitory electronic memory 224 and/or the other components are included within a single device. In other embodiments, the processor 222 and the non-transitory electronic memory 224 and/or the other components may be distributed among multiple devices that are communicatively coupled. The controller 220 includes non-transitory electronic memory 224 that stores a set of machine-readable instructions. The processor 222 executes the machine-readable instructions stored in the non-transitory electronic memory 224. The non-transitory electronic memory 224 may comprise RAM, ROM, flash memories, hard drives, or any device capable of storing machine-readable instructions such that the machine-readable instructions can be accessed by the processor 222. Accordingly, the operations system 200 described herein can be implemented in any conventional computer programming language, as pre-programmed hardware elements, or as a combination of hardware and software components. The non-transitory electronic memory 224 can be implemented as one memory module or a plurality of memory modules. In some embodiments, the non-transitory electronic memory 224 includes instructions for executing the functions of the operations system 200.

The processor 222 can be any device capable of executing machine-readable instructions. For example, the processor 222 can be an integrated circuit, a microchip, a computer, or any other computing device. The non-transitory electronic memory 224 and the processor 222 are coupled to the communication path 211 that provides signal interconnectivity between various components and/or modules of the operations system 200. Accordingly, the communication path 211 can communicatively couple any number of processors with one another, and allow the modules coupled to the communication path 211 to operate in a distributed computing environment. Specifically, each of the modules can operate as a node that can send and/or receive data. As used herein, the term “communicatively coupled” means that coupled components are capable of exchanging data signals with one another such as, for example, electrical signals via conductive medium, electromagnetic signals via air, optical signals via optical waveguides, and the like. The communication path 211 can be at least partially formed in or on the primary conductive plate 136.

As schematically depicted in FIG. 9, the communication path 211 communicatively couples the processor 222 and the non-transitory electronic memory 224 of the controller 220 with a plurality of other components of the operations system 200. For example, the operations system 200 depicted in FIG. 9 includes the processor 222 and the non-transitory electronic memory 224 communicatively coupled with the network interface hardware 214 and the battery 138.

The battery 138 provides power to the one or more components of the onboard control unit 210 and/or the dosing module 100. In some embodiments, the battery 138 is a rechargeable direct current power source.

In some embodiments, the operations system 200 can include a display device 212. The display device 212 is coupled to the communication path 211 such that the communication path 211 communicatively couples the display device 212 to other modules of the operations system 200. The display device 212 can be located on the dosing module 100, for example, as part of the onboard control unit 210, and can output a notification in response to a determination of a size of an administered dose of medication. In some embodiments, the display device 212 can display other sensor measurements, such as indications that an administration of a dose has begun, the charge remaining in the battery 138, and/or the number of times the dosing module 100 has been connected to one or more delivery pens 10.

In some embodiments, the operations system 200 includes network interface hardware 214 for communicatively coupling the onboard control unit 210 to a remote device 228 via a network 226. The remote device 228 can include, without limitation, a smartphone, a tablet, a personal media player, or any other electric device that includes wireless communication functionality. It is to be appreciated that, when provided, the remote device 228 can serve to provide user notifications instead of or in addition to the display device 212. The onboard control unit 210 can be communicatively coupled with the remote device through Bluetooth 2.0, near-field communication (NFC), ZigBee, or other suitable protocol.

As noted above, in some embodiments, the non-transitory electronic memory 224 includes instructions for executing the functions of the operations system 200. The instructions can include instructions for detecting when an injection of a dose begins. For instance, in some embodiments, the instructions can include instructions for the controller 220 to gather a signal from the circuit board 148 (FIG. 5) (or the conductive spring clips 146 (FIG. 5) or other sensors coupled to the button) and determine from the signal that a dose injection has been initiated. In some embodiments, the instructions can include instructions for the controller 220 to, once it is determined that a dose injection has been initiated, gather a signal from the contact clips 122 (FIG. 5) or one or more other sensors configured to sense rotation of the encoder wheel 118 (FIG. 5) (via the secondary conductive plate 120 (FIG. 5), for instance) and determine from the signal the degree of rotation of the lead screw 7 (FIG. 2) and/or inner sleeve 25 (FIG. 3C) of the delivery pen 10 from the rotation of the encoder wheel 118 (FIG. 5). In some embodiments, the instructions can include instructions for the controller 220 to, based on the determined degree of rotation of the lead screw 7 (FIG. 2), inner sleeve 25 (FIG. 3C), and/or encoder wheel 118 (FIG. 5) and other delivery pen 10 data, such as the size of the medicament cartridge 12 (FIG. 2) or amount of medication in the medicament cartridge 12, determine the size of the dose of medication delivered during an injection.

In some embodiments, the instructions can include instructions for the controller 220 to, once the size of the dose is determined, display the dose size on the display device 212 or communicate the dose size to the remote device 228 for display on the remote device 228. In some embodiments, the instructions can include instructions for the controller 220 to gather a signal from the battery 138, determine a battery life or charge of the battery 138 based at least in part on the signal, and display the charge on the display device 212 or communicate the charge to the remote device 228 for display on the remote device 228. In some embodiments, the instructions can include instructions for the controller 220 to gather a signal from the sensor 190 (FIG. 5), determine, based at least in part on the signal, the number of times the dosing module 100 has been attached to a delivery pen 10, when the delivery pen 10 was last changed, how much medicament remains in the medicament cartridge 12 (FIG. 2), and/or the age of medicament in the medicament cartridge 12 (FIG. 2), and display the determination on the display device 212 or communicate the determination to the remote device 228 for display on the remote device 228.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification, embodiments have been described in a way which enables a clear and concise specification to be written, but it is intended and will be appreciated that embodiments may be variously combined or separated without parting from the scope of the present disclosure. It is intended that the present disclosure be limited only to the extent required by the appended claims and the applicable rules of law.

As utilized herein, the terms “comprise” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one nonlimiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A dosing module, comprising:
an outer shell, the outer shell comprising a stem;
a spline hole plate having a spline hole, wherein the spline hole plate is positioned within the stem of the outer shell and sized to receive a spline shaft extending proximally from a shaft spring, such that the spline hole plate and the shaft spring are rotatable together;
an encoder wheel rigidly coupled to the spline hole plate and positioned within the stem of the outer shell;
one or more sensors positioned within the stem of the outer shell and configured to detect a rotation of the encoder wheel, wherein the encoder wheel is configured to rotate with respect to the one or more sensors, wherein:
the dosing module is removably couplable to a dose knob of a delivery pen, wherein the stem is received within an internal well of the dose knob when the dosing module is coupled to the dose knob;
the shaft spring is directly coupled to a component of a drive mechanism of the delivery pen; and
the dosing module is configured to measure a size of a dose administered from the delivery pen.

2. The dosing module of claim 1, wherein the dosing module is removably couplable to the dose knob when a first button originally coupled to the delivery pen is removed from the delivery pen.

3. The dosing module of claim 2, where the first button, when coupled to the delivery pen, is configured to initiate administration of a dose from the delivery pen when the first button is depressed.

4. The dosing module of claim 1, wherein:
the stem has a peripheral edge; and
the peripheral edge of the stem is configured to contact a radially inner surface of the internal well when the dosing module is coupled to the dose knob.

5. The dosing module of claim 1, wherein:
the stem comprises a peripheral surface and a retaining ridge extending radially outward from the peripheral surface of the stem; and
the retaining ridge is configured to be received in a groove along a radially inner surface of the internal well when the dosing module is coupled to the dose knob.

6. The dosing module of claim 1, wherein:
the outer shell further comprises a body having a peripheral edge; and
an outer dimension of the peripheral edge of the body of the outer shell is equal to or less than an outer dimension of a peripheral edge of the dose knob.

7. The dosing module of claim 6, wherein the outer dimension of the peripheral edge of the body of the outer shell is greater than an outer dimension of a peripheral edge of the stem of the outer shell.

8. The dosing module of claim 1, further comprising:
a button;
a circuit board; and
one or more conductive spring clips, wherein each of the one or more conductive spring clips have a first end coupled to the circuit board and a second free end, wherein:
the button is configured to initiate administration of a dose from the delivery pen when the button is depressed; and
when the button is depressed, the button is configured to deform the one or more conductive spring clips such that the one or more conductive spring clips complete a circuit.

9. The dosing module of claim 8, further comprising:
an inner shell positioned within an interior of the outer shell, wherein:
the spline hole plate is coupled to the outer shell such that the spline hole plate and the outer shell are rotatable together;
the button is positioned at least partially within the inner shell; and
the outer shell is rotatable relative to the inner shell.

10. A dosing module, comprising:
an outer shell, the outer shell comprising a body and a stem, wherein:
the stem is sized to be received within an internal well of a dose knob of a delivery pen; and
an outer dimension of a peripheral edge of the body of the outer shell is equal to or less than an outer dimension of a peripheral edge of the dose knob, wherein:
the dosing module is removably couplable to the dose knob of the delivery pen; and
the dosing module is configured to measure a size of a dose administered from the delivery pen.

11. The dosing module of claim 10, wherein the outer dimension of the peripheral edge of the body of the outer shell is greater than an outer dimension of a peripheral edge of the stem of the outer shell.

12. The dosing module of claim 10, wherein the body of the outer shell further comprises a distal end wall having a distal surface, wherein the distal surface of the distal end wall is configured to contact a proximal surface of a proximal end wall of the dose knob when the dosing module is coupled to the dose knob.

13. The dosing module of claim 10, wherein:
the stem comprises a peripheral edge; and
the peripheral edge of the stem is configured to contact a radially inner surface of the internal well when the dosing module is coupled to the dose knob.

14. The dosing module of claim 10, wherein:
the stem comprises a peripheral surface and a retaining ridge extending radially outward from the peripheral surface of the stem; and
the retaining ridge is configured to be received in a groove along a radially inner surface of the internal well when the dosing module is coupled to the dose knob.

15. The dosing module of any one of claims 10-14, wherein the stem comprises a distal wall having an opening configured to receive a shaft coupled to a drive mechanism of the delivery pen.

16. The dosing module of claim 15, wherein the opening in the distal wall of the stem is chamfered or beveled.

17. The dosing module of claim 10, further comprising a sensor coupled to the stem, wherein the sensor is configured to detect when the stem is received within the internal well.

18. A dosing module, comprising:
a spline hole plate having a spline hole, wherein the spline hole is sized to receive a spline shaft extending proximally from a shaft spring, such that the spline hole plate and the shaft spring are rotatable together; and
an encoder wheel rigidly coupled to the spline hole plate, wherein:
the shaft spring is coupled to a component of a drive mechanism of a delivery pen;
the dosing module is removably couplable to the delivery pen; and
the dosing module is configured to measure a size of a dose administered from the delivery pen.

19. The dosing module of claim 18, further comprising one or more sensors configured to detect a rotation of the encoder wheel, wherein the encoder wheel is configured to rotate with respect to the one or more sensors.

20. The dosing module of claim 19, wherein:
the encoder wheel comprises one or more electrically conductive pads; and
the one or more sensors comprise contact clips configured to contact the one or more electrically conductive pads.

21. The dosing module of claim 19, wherein:
the encoder wheel comprises one or more magnetic poles; and
the one or more sensors comprise one or more magnetic sensors.

22. The dosing module of claim 19, wherein:
the encoder wheel comprises one or more markings; and
the one or more sensors comprise one or more optical sensors.

23. The dosing module of any one of claims 18-22, further comprising a button, wherein the button is configured to initiate administration of a dose from the delivery pen when the button is depressed.

24. The dosing module of claim 23, further comprising:
a circuit board; and
one or more conductive spring clips, wherein each of the one or more conductive spring clips have a first end coupled to the circuit board and a second free end, wherein:
when the button is depressed, the button is configured to deform the one or more conductive spring clips such that the one or more conductive spring clips complete a circuit.

25. The dosing module of claim 24, further comprising:
an outer shell; and
an inner shell positioned within an interior of the outer shell, wherein:
the spline hole plate is coupled to the outer shell such that the spline hole plate and the outer shell are rotatable together;
the button is positioned at least partially within the inner shell; and
the outer shell is rotatable relative to the inner shell.

26. A delivery pen system, comprising:
a delivery pen, comprising:
a dose knob at a proximal end of the delivery pen, wherein the dose knob comprises an internal well; and
a proximally extending spline shaft positioned in the internal well; and
a dosing module removably couplable to the dose knob and configured to measure a size of a dose administered from the delivery pen, wherein the dosing module comprises a spline hole plate having a spline hole sized to receive the proximally extending spline shaft such that the spline hole plate and the spline shaft are rotatable together.

27. The delivery pen system of claim 26, wherein the delivery pen further comprises:
a drive mechanism for delivering a dose, the drive mechanism comprising an inner sleeve; and
a shaft spring positioned in the internal well, wherein:
the spline shaft extends proximally from the shaft spring; and
the inner sleeve is coupled to the shaft spring such that the inner sleeve and the shaft spring are rotatable together.

28. The delivery pen system of claim 27, wherein at least a portion of the inner sleeve is positioned in the internal well.

29. The delivery pen system of claim 28, wherein:
a proximal surface of a distal end wall of the dose knob and a distal surface of a flange of the inner sleeve comprise corresponding features that are configured to mate to allow the dose knob and the inner sleeve to rotate together.

30. The delivery pen system of claim 29, wherein the corresponding features comprise corresponding ribbing.

31. The delivery pen system of claim 27, wherein the inner sleeve and the shaft spring comprise corresponding features that are configured to mate to allow the inner sleeve and the shaft spring to rotate together.

32. The delivery pen system of claim 31, wherein the corresponding features comprise:
one or more pegs extending proximally from a flange of the inner sleeve; and
one or more bores in a surface of the shaft spring, the one or more bores configured to receive the one or more pegs.

33. The delivery pen system of claim 26, wherein the spline hole is chamfered or beveled.

34. The delivery pen system of any one of claims 26-33, wherein the dosing module further comprises:
an encoder wheel coupled to the spline hole plate such that the encoder wheel and spline hole plate are rotatable together; and
one or more sensors configured to detect a rotation of the encoder wheel.

35. The delivery pen system of claim 34, wherein:
the encoder wheel comprises one or more electrically conductive pads; and
the one or more sensors comprise contact clips configured to contact the one or more electrically conductive pads, wherein the encoder wheel is configured to rotate relative the contact clips.

36. The delivery pen system of claim 34, wherein:
the encoder wheel comprises one or more magnetic poles; and
the one or more sensors comprise one or more magnetic sensors, wherein the encoder wheel is configured to rotate relative the one or more magnetic sensors.

37. The delivery pen system of claim 34, wherein:
the encoder wheel comprises one or more markings; and
the one or more sensors comprise one or more optical sensors, wherein the encoder wheel is configured to rotate with respect to the one or more optical sensors.

38. The delivery pen system of claim 26, wherein the dosing module further comprises a button, wherein the button is configured to initiate administration of a dose from the delivery pen when the button is depressed.

39. The delivery pen system of claim 38, wherein the dosing module further comprises:
a circuit board; and
one or more conductive spring clips, wherein each of the one or more conductive spring clips have a first end coupled to the circuit board and a second free end, wherein:
when the button is depressed, the button deforms the one or more conductive spring clips such the one or more conductive spring clips complete a circuit.

40. The delivery pen system of claim 38 or 39, further comprising:
an outer shell; and
an inner shell positioned within an interior of the outer shell, wherein:
the spline hole plate is coupled to the outer shell such that the spline hole plate and the outer shell are rotatable together;
the button is positioned at least partially within the inner shell; and
the outer shell is rotatable relative to the inner shell.

41. The delivery pen system of claim 26, further comprising a button removably couplable to the dose knob, wherein the button, when coupled to the delivery pen, is configured to initiate administration of a dose from the delivery pen when the button is depressed.

42. The delivery pen system of claim 41, wherein the dosing module is couplable to the dose knob when the button is removed from the dose knob.

43. A delivery pen system, comprising:
a delivery pen, comprising a dose knob at a proximal end of the delivery pen, wherein the dose knob comprises:
an internal well; and
a peripheral edge; and
a dosing module removably couplable to the dose knob and configured to measure a size of a dose administered from the delivery pen, wherein the dosing module comprises an outer shell, the outer shell comprising a body and a stem, wherein:
the stem is sized to be received within the internal well; and
an outer dimension of a peripheral edge of the body of the outer shell is equal to or less than an outer dimension of the peripheral edge of the dose knob.

44. The delivery pen system of claim 43, wherein the outer dimension of the peripheral edge of the body of the outer shell is greater than an outer dimension of a peripheral edge of the stem of the outer shell.

45. The delivery pen system of claim 43, wherein:
the dose knob comprises a proximal end wall having a proximal surface;
the body of the outer shell of the dosing module comprises a distal end wall having a distal surface; and
the distal surface of the distal end wall of the body is configured to contact the proximal surface of the proximal end wall of the dose knob when the dosing module is coupled to the dose knob.

46. The delivery pen system of any one of claims 43-45, wherein:
the internal well comprises a radially inner surface;
the stem comprises a peripheral edge; and
the radially inner surface of the internal well is configured to contact the peripheral edge of the stem when the dosing module is coupled to the dose knob.

47. The delivery pen system of claim 46, wherein contact between the radially inner surface of the internal well and the peripheral edge of the stem when the dosing module is coupled to the dose knob is configured to create a friction fit between the dosing module and the dose knob.

48. The delivery pen system of claim 43, wherein:
the internal well comprises a radially inner surface and a groove along the radially inner surface;
the stem comprises a peripheral surface and a retaining ridge extending radially outward from the peripheral surface of the stem; and
the retaining ridge is configured to be received in the groove when the dosing module is coupled to the dose knob.

49. The delivery pen system of claim 48, wherein the retaining ridge and the groove are configured to form a snap fit when the dosing module is coupled to the dose knob.

50. The delivery pen system of claim 43, wherein:
the dose knob comprises a shaft coupled to a drive mechanism of the delivery pen;
the stem comprises a distal wall having an opening; and
the shaft is received in the opening in the distal wall of the stem when the dosing module is coupled to the dose knob.

51. The delivery pen system of claim 50, wherein the opening in the distal wall of the stem is chamfered or beveled.

52. The delivery pen system of claim 43, wherein the dosing module further comprises a sensor coupled to the stem, wherein the sensor is configured to detect when the stem is received within the internal well.

53. The delivery pen system of claim 43, further comprising a button removably couplable to the dose knob, wherein the button, when coupled to the delivery pen, is configured to initiate administration of a dose from the delivery pen when the button is depressed.

54. The delivery pen system of claim 53, wherein the dosing module is couplable to the dose knob when the button is removed from the dose knob.

55. A delivery pen system, comprising:
a delivery pen, comprising:
a dose knob at a proximal end of the delivery pen, wherein the dose knob comprises an internal well and a proximal surface at a distal end wall of the internal well;
a shaft spring positioned in the internal well, the shaft spring comprising a spline shaft extending proximally therefrom; and
a drive mechanism for delivering a dose, the drive mechanism comprising an inner sleeve comprising a flange having a proximal surface and a distal surface, wherein:
the proximal surface of the flange of the inner sleeve and the shaft spring comprise corresponding features that are configured to mate to allow the inner sleeve and the shaft spring to rotate together; and
the proximal surface of the distal end wall of the internal well and the distal surface of the flange of the inner sleeve comprise corresponding features that are configured to mate to allow the dose knob and the inner sleeve to rotate together; and
a dosing module removably couplable to the dose knob and configured to measure a size of a dose administered from the delivery pen, wherein the dosing module comprises:
a spline hole plate having a spline hole sized to receive the spline shaft such that the spline hole plate and the spline shaft are rotatable together;
an encoder wheel coupled to the spline hole plate such that the encoder wheel and spline hole plate are rotatable together, wherein the encoder wheel comprises one or more electrically conductive pads; and
one or more contact clips configured to contact the one or more electrically conductive pads, wherein the encoder wheel is configured to rotate relative the one or more contact clips.

56. A delivery pen system, comprising:
a delivery pen, comprising a dose knob at a proximal end of the delivery pen;
a button removably couplable to the dose knob; and
a dosing module removably couplable to the dose knob and configured to measure a size of a dose administered from the delivery pen.

57. A system, comprising:
a delivery pen, comprising:
a dose knob at a proximal end of the delivery pen, wherein the dose knob comprises an internal well;
a drive mechanism for delivering a dose; and
a proximally extending spline shaft positioned in the internal well, wherein the spline shaft is coupled to a component of the drive mechanism such that the spline shaft and the component of the drive mechanism are rotatable together;
a dosing module removably couplable to the dose knob, wherein the dosing module comprises:
a spline hole plate having a spline hole sized to receive the proximally extending spline shaft such that the spline hole plate and the spline shaft are rotatable together;
an encoder wheel coupled to the spline hole plate such that the encoder wheel and spline hole plate are rotatable together; and
one or more sensors configured to sense a rotation of the encoder wheel, wherein the encoder wheel is configured to rotate with respect to the one or more sensors; and
a control system configured to:
receive sensor data from the one or more sensors;
determine a degree of rotation of the encoder wheel based on the sensor data; and
determine a size of a dose delivered from the delivery pen during an injection.

58. The system of claim 57, wherein:
the encoder wheel comprises one or more electrically conductive pads; and
the one or more sensors comprise contact clips configured to contact the one or more electrically conductive pads.

59. The system of claim 57, wherein:
the encoder wheel comprises one or more magnetic poles; and
the one or more sensors comprise one or more magnetic sensors.

60. The system of claim 57, wherein:
the encoder wheel comprises one or more markings; and
the one or more sensors comprise one or more optical sensors.

61. The system of any one of claims 57-60, wherein the control system is further configured to determine the size of the dose delivered from the delivery pen during the injection based, at least in part, on the degree of rotation of the encoder wheel.

62. The system of any one of claims 57-60, wherein the control system is further configured to determine a degree of rotation of one or more components of the drive mechanism of the delivery pen based on the degree of rotation of the encoder wheel.

63. The system of claim 62, wherein the control system is further configured to determine the size of the dose delivered from the delivery pen during the injection based, at least in part, on the degree of rotation of the one or more components of the drive mechanism.

64. The system of claim 57, wherein:
the dosing module further comprises:
a button configured to initiate administration of a dose from the delivery pen when the button is depressed;
a circuit board; and
one or more conductive spring clips, wherein each of the one or more conductive spring clips have a first end coupled to the circuit board and a second free end, wherein:
when the button is depressed, the button is configured to deform the one or more conductive spring clips such that the one or more conductive spring clips complete a circuit; and
the control system is further configured to:
receive a signal indicating the circuit is complete; and
determine that the injection has been initiated based on the signal indicating the circuit is complete.

65. The system of claim 64, wherein the control system is further configured to:
receive sensor data from the one or more sensors and determine the degree of rotation of the encoder wheel based on the sensor data for as long as the circuit is complete.

66. The system of claim 64, wherein the control system is further configured to:
receive sensor data from the one or more sensors and determine the degree of rotation of the encoder wheel based on the sensor data for a set period of time after receiving the signal indicating the circuit is complete.

67. The system of claim 57, wherein the control system is further configured to display the size of the dose delivered on a display of the dosing module.

68. The system of claim 57, wherein the control system is further configured to display the size of the dose delivered on a display external to the dosing module.

69. The system of claim 57, wherein the control system is internal to the dosing module.

70. The system of claim 57, wherein:
the dosing module further comprises a second sensor configured to sense when the dosing module is received in the internal well of the dose knob; and
the control system is further configured to:
receive sensor data from the second sensor; and
determine when the dosing module was last coupled to a new delivery pen based on the sensor data from the second sensor.

71. The system of claim 57, wherein:
the dosing module further comprises a second sensor configured to sense when the dosing module is received in the internal well of the dose knob; and
the control system is further configured to:
receive sensor data from the second sensor; and
determine an amount of medicament left in the delivery pen based on the sensor data from the second sensor and the size of the dose delivered from the delivery pen during the injection.

72. A method, comprising:
sensing, with at least one sensor of a dosing module, data regarding a rotation of an encoder wheel of the dosing module, wherein:
the encoder wheel is rotatable with a spline shaft of a delivery pen; and
the spline shaft is coupled to a component of a drive mechanism of the delivery pen, wherein the drive mechanism is configured to deliver a dose from the delivery pen;
sending, by the at least one sensor, the sensed data to a controller;
identifying, by the controller and from the sensed data, a degree of rotation of the encoder wheel; and
identifying, by the controller, a size of a dose delivered from the delivery pen during an injection.

73. The method of claim 72, wherein the size of the dose delivered from the delivery pen during the injection is determined from the degree of rotation of the encoder wheel.

74. The method of claim 72, further comprising:
identifying, by the controller and from the degree of rotation of the encoder wheel, a degree of rotation of one or more components of the drive mechanism.

75. The method of claim 74, wherein the size of the dose delivered from the delivery pen during the injection is determined from the degree of rotation of the one or more components of the drive mechanism.

76. The method of claim 72, further comprising displaying the size of the dose delivered from the delivery pen during the injection on a display of the dosing module.

77. The method of claim 72, further comprising displaying the size of the dose delivered from the delivery pen during the injection on a display external to the dosing module.

78. The method of claim 72, further comprising:
identifying, by the controller, a time when the injection of the dose is initiated; and
identifying, by the controller, the degree of rotation of the encoder wheel for a set period of time after the injection of the dose is initiated.

79. The method of claim 72, further comprising:
identifying, by the controller, when a button of the dosing module is depressed, wherein the button is configured to initiate administration of the dose from the delivery pen when the button is depressed; and
identifying, by the controller, the degree of rotation of the encoder wheel for as long as the button is depressed.

80. The method of any one of claims 72-79, further comprising:
sensing, with a second sensor of the dosing module, data regarding a coupling of the dosing module to the delivery pen;
sending, by the second sensor, the sensed data to the controller; and
identifying, by the controller and from the sensed data from the second sensor and the size of the dose delivered from the delivery pen during the injection, an amount of medicament left in the delivery pen.
